(19) United States
(12) Patent Application Publication
(10) Pub. No.: US 2017/0119347 A1
(43) Pub. Date: May 4, 2017

(54) ROBOTIC SYSTEMS FOR CONTROL OF AN ULTRASONIC PROBE

(71) Applicant: Neural Analytics, Inc., Los Angeles, CA (US)

(72) Inventors: Roman Flores, II, Los Angeles, CA (US); Matthew Hutter, Los Angeles, CA (US); Gerard Salinas, Los Angeles, CA (US); Michael Costa, Los Angeles, CA (US); Matthew Sylvester, Los Angeles, CA (US); Robert Hamilton, Los Angeles, CA (US); Corey Thibeault, Los Angeles, CA (US); Leo Petrossian, Los Angeles, CA (US); Shankar Radhakrishnan, Los Angeles, CA (US); Michael O'Brien, Los Angeles, CA (US); Seth Wilk, Los Angeles, CA (US); Jan Zwierstra, Los Angeles, CA (US)

(73) Assignee: Neural Analytics, Inc., Los Angeles, CA (US)

(21) Appl. No.: 15/399,648

(22) Filed: Jan. 5, 2017

Related U.S. Application Data
(63) Continuation-in-part of application No. 15/187,397, filed on Jun. 20, 2016.
(60) Provisional application No. 62/275,192, filed on Jan. 5, 2016.
(60) Provisional application No. 62/181,859, filed on Jun. 19, 2015.
(60) Provisional application No. 62/181,862, filed on Jun. 19, 2015.
(60) Provisional application No. 62/347,527, filed on Jun. 8, 2016.

(51) Int. Cl. A61B 8/00; A61B 8/06; A61B 8/08; B25J 9/16; B25J 13/08

(52) U.S. Cl. CPC A61B 8/4461; B25J 9/1689; B25J 13/085; B25J 9/1633; A61B 8/0808; A61B 8/06; A61B 8/488

(57) ABSTRACT

According to various embodiments, there is provided a headset mountable on a head, the headset including a probe for emitting energy into the head. The headset further includes a support structure coupled to the probe. The support structure includes translation actuators for translating the probe along axes about a surface of the head.

### CROSS-REFERENCE TO RELATED PATENT APPLICATIONS

This application is a continuation in part of U.S. patent application Ser. No. 15/187,397, filed Jun. 20, 2016 and now pending, titled TRANSCRANIAL DOPPLER PROBE, which claims priority to, and the benefit of, U.S. provisional patent application Ser. No. 62/181,859, titled AUTOMATIC DISCOVERY OF TRANSCRANIAL DOPPLER WINDOW, and filed on Jun. 19, 2015, which is incorporated herein by reference in its entirety, and which also claims priority to, and the benefit of, U.S. provisional patent application Ser. No. 62/181,862, titled INITIAL PLACEMENT OF TRANSCRANIAL DOPPLER SENSORS, and filed on Jun. 19, 2015, which is incorporated herein by reference in its entirety, and which also claims priority to, and the benefit of, U.S. provisional patent application Ser. No. 62/347,527, titled PROBE SUPPORT STRUCTURE WITH VARIABLE STIFFNESS, and filed on Jun. 8, 2016, which is incorporated herein by reference in its entirety. The present disclosure claims priority to, and the benefit of, U.S. provisional patent application Ser. No. 62/275,192, titled SYSTEMS AND METHODS FOR DETECTING NEUROLOGICAL CONDITIONS, and filed on Jan. 5, 2016, which is incorporated herein by reference in its entirety.

### FIELD

Subject matter described herein relates generally to medical devices, and more particularly to a headset including a probe for diagnosing medical conditions.

### BACKGROUND

Transcranial Doppler (TCD) is used to measure the cerebral blood flow velocity (CBFV) in the major conducting arteries of the brain (e.g., the Circle of Willis) non-invasively. It is used in the diagnosis and monitoring a number of neurologic conditions, such as the assessment of arteries after a subarachnoid hemorrhage (SAH), aiding preventative care in children with sickle cell anemia, and risk assessment in embolic stroke patients or subjects.

Traditionally, a TCD ultrasound includes the manual positioning of a probe relative to a patient or subject by a technician. The probe emits energy into the head of a patient or subject. The technician identifies the CBFV waveform signature of a cerebral artery or vein in the head. Identification of the signal requires integration of probe insonation depth, angle, and placement within one of several ultrasound windows as well as characteristics from the ultrasound signal which include waveform spectrum, sounds, M-Mode, and velocity. For devices utilizing a probe (e.g., an automated Transcranial Doppler device), there exist concerns related to alignment and pressure that the probe exerts during use (e.g., for comfortability and safety when held against a human being or for ensuring the effectiveness of the probe). In some devices, a spring is incorporated within a probe, but such devices may not be effective for pressure control due to lateral slippage and shifting of the spring within the probe.

### SUMMARY

According to various embodiments, there is provided a headset mountable on a head, the headset including a probe for emitting energy into the head. The headset may further include a support structure coupled to the probe, with the support structure including translation actuators for translating the probe along at least two axes generally parallel to a surface of the head.

In some embodiments, the headset may further include at least a perpendicular translation actuator for translating the probe along a perpendicular axis generally perpendicular to the surface of the head. In some embodiments, the headset may further include at least one rotation actuator for rotating the probe about at least one rotation axis. The headset may further include a tilt axis generally orthogonal to the perpendicular axis. The headset may further include a pan axis generally orthogonal to the perpendicular axis.

In some embodiments, the headset may provide exactly five actuated degrees of freedom of movement of the probe including two actuated degrees of freedom of translation through the two axes generally parallel to the surface of the head (x,y), one actuated degree of freedom through the perpendicular axis generally perpendicular to the surface of the head (z), one actuated degree of freedom along the tilt axis, and one actuated degree of freedom along the pan axis.

According to various embodiments, there is provided a device configured to interact with a target surface, the device including a probe configured to interact with the target surface. The device may further include a support structure coupled to the probe for moving the probe relative to the target surface. The support structure may be configured to translate the probe along both a translation plane generally parallel to the target surface. The support structure may be further configured to rotate the probe about at least one rotation axis.

In some embodiments, the support structure is configured to translate the probe along a translation axis generally perpendicular to the translation plane. In some embodiments, the support structure includes a tilt axis different than the translation axis. In some embodiments, the support structure includes a pan axis different than the translation axis and the tilt axis. In some embodiments, the support structure is further configured to rotate the probe towards and away from the target surface about the tilt axis and the pan axis. In some embodiments, the support structure has a stiffness along each of the translation plane and the translation axis, and the stiffness along the translation plane is greater than the stiffness along the translation axis. In some embodiments, the probe is configured to emit ultrasound waves into the target surface.

In some embodiments, the device further includes a first actuator configured to translate the probe along a first direction along the translation plane. In some embodiments, the device further includes a second actuator configured to translate the probe along a second direction perpendicular to the first direction along the translation plane. In some embodiments, the device further includes a third actuator configured to translate the probe along the translation axis perpendicular to the translation plane. In some embodiments, the first actuator and the second actuator are configured with a stiffness of the translation plane, and the third actuator is configured with a stiffness of the translation axis.

In some embodiments, the first, second, and third actuators are a servo motor.

In some embodiments, an input force of each of the first, second, and third actuators is determined by a method including determining a configuration of the support structure for the probe and each of the first, second, and third actuators for the support structure. In some embodiments, the method further includes determining a stiffness matrix for the support structure based on the configuration of the support structure and a desired conditional stiffness of the support structure. In some embodiments, the method further includes determining a force vector by multiplying the stiffness matrix and a vector of a difference of the desired and actual translational and rotational position of the probe. In some embodiments, the method further includes calculating a Jacobian for the support structure. In some embodiments, the method further includes determining the input forces for each of the first, second, and third actuators by multiplying the force vector and a transpose of the Jacobian.

According to various embodiments, there is provided a method of manufacturing a device configured to interact with a target surface, including providing a probe configured to interact with the target surface. In some embodiments, the method further includes coupling a support structure to the probe for moving the probe relative to the target surface, wherein the support structure configured to translate the probe along both a translation plane generally parallel to the target surface and along a translation axis generally perpendicular to the translation plane and rotate the probe about at least one rotation axis. In some embodiments, the one rotation axis includes a tilt axis different than the translation axis. In some embodiments the one rotation axis includes a pan axis different than the translation axis and the tilt axis.

According to various embodiments, there is provided a robotic system for use in scanning a subject, the robotic system including a probe for emitting energy into the subject, a robotic support structure coupled to the probe, the robotic support structure including actuators for moving the probe parallel to a surface of the subject. In some embodiments, the robotic system includes a robotic support structure with five actuated degrees of freedom. In some embodiments, the robotic system includes a robotic support structure with six actuated degrees of freedom. In some embodiments, the robotic system includes a robotic support structure with more than six actuated degrees of freedom. In some embodiments, the robotic system includes a robotic support structure with more than six actuated degrees of freedom. In some embodiments, the robotic system includes a robotic support structure with four actuated degrees of freedom. In some embodiments, the robotic system includes a control computer configured to control movement of the robotic support structure. In some embodiments, the robotic system includes a teleoperated controller configured to control movement of the robotic support structure. In some embodiments, the robotic system includes a hybrid position-force controller configured to control movement of the robotic support structure. In some embodiments, the robotic system includes a force/torque sensor in contact with the probe.

According to various embodiments, there is provided a device configured to interact with a target surface, including a probe configured to interact with the target surface, and a support structure coupled to the probe for moving the probe relative to the target surface, the support structure, including a hybrid position-force controller that controls movement of the support structure. In some embodiments, the hybrid position-force controller includes a spring configured to press the probe against the target surface to maintain contact force passively. In some embodiments, the hybrid position-force controller includes a first motor configured to move the probe along a first axis. In some embodiments, the hybrid position-force controller includes a second motor configured to move the probe along a second axis. In some embodiments, the hybrid position-force controller includes a third motor configured to rotate the probe about a third axis. In some embodiments, the hybrid position-force controller includes a fourth motor configured to rotate the probe about a fourth axis.

According to various embodiments, there is provided an automated TCD system including a TCD probe configured to insonate a vessel of a patient, a robot mounted to the probe, and a computer connected to the robot, which computer controls the movement of the robot. In some embodiments, the automated TCD system includes an endeffector with an axial force sensor mounted to the robot in communication with the probe. In some embodiments, the automated TCD system includes a robot configured to move with at least six actuated degrees of freedom. In some embodiments, the automated TCD system includes a robot configured to move with exactly five actuated degrees of freedom. In some embodiments, the automated TCD system includes a robot configured to move with exactly four actuated degrees of freedom.

### BRIEF DESCRIPTION OF THE DRAWINGS

Features, aspects, and advantages of the present invention will become apparent from the following description and the accompanying exemplary embodiments shown in the drawings, which are briefly described below.

FIG. 1 is a diagram of a virtual support structure for manipulating a medical probe, according to an exemplary embodiment.

FIG. 2 is an perspective view of a medical probe and a gimbal structure, according to an exemplary embodiment.

FIG. 3 is a perspective view of a two-link revolute support structure for the medical probe of FIG. 2, according to an exemplary embodiment.

FIG. 4 is front elevation view of the support structure of FIG. 3.

FIG. 5 is a right side elevation view of the support structure of FIG. 3.

FIG. 6 is a perspective view of a prismatic support structure for the medical probe of FIG. 2, according to an exemplary embodiment.

FIG. 7 is front elevation view of the support structure of FIG. 6.

FIG. 8 is a right side elevation view of the support structure of FIG. 6.

FIG. 9 is a schematic front view diagram of the support structure of FIG. 3.

FIG. 10 is a schematic front view diagram of the support structure of FIG. 6.

FIG. 11 is a flowchart of a method for determining the input force, or torque, for an actuator, according to an exemplary embodiment.

FIG. 12 is a perspective view of a 5-bar parallel mechanism (revolute-revolute) support structure for the medical probe of FIG. 2, according to an exemplary embodiment.

FIG. 13 is front elevation view of the support structure of FIG. 12.

FIG. 14 is a right side elevation view of the support structure of FIG. 12.

FIG. 15 illustrates a hybrid position-force admittance controller.

FIG. 16 illustrates a probe on a redundant manipulator.

FIG. 17 illustrates a probe on a redundant manipulator mounted on a monitoring station.

FIG. 18 illustrates a probe on a redundant manipulator scanning through the zygomatic arch.

FIG. 19 illustrates a probe on a redundant manipulator performing a transorbital scan through an eye socket or orbit.

FIG. 20 illustrates a probe on a redundant manipulator scanning through the occipital bone.

FIG. 21 illustrates a probe on a redundant manipulator scanning through the mandibular.

FIG. 22 illustrates a schematic diagram of a TCD system.

FIG. 23A illustrates a probe on a redundant manipulator.

FIG. 23B illustrates test results of force output for a probe on a redundant manipulator.

FIG. 24 illustrates a top perspective view of a spring loaded probe in a support structure with four actuated degrees of freedom as well as one passive degree of freedom.

FIG. 25 illustrates a front perspective view of a spring loaded probe in a support structure with four actuated degrees of freedom as well as one passive degree of freedom.

FIG. 26 illustrates a cross-sectional view of a spring loaded probe in a support structure with four actuated degrees of freedom as well as one passive degree of freedom.

FIG. 27 illustrates a front perspective view of a five actuated degrees of freedom prismatic support structure for a medical probe, according to an exemplary embodiment.

FIG. 28 illustrates a rear perspective view of a five actuated degrees of freedom prismatic support structure for the a medical probe, according to an exemplary embodiment.

FIG. 29 illustrates an exploded perspective view of a five actuated degrees of freedom prismatic support structure for the a medical probe, according to an exemplary embodiment.

### DETAILED DESCRIPTION

The detailed description set forth below in connection with the appended drawings is intended as a description of various configurations and is not intended to represent the only configurations in which the concepts described herein may be practiced. The detailed description includes specific details for providing a thorough understanding of various concepts. However, it will be apparent to those skilled in the art that these concepts may be practiced without these specific details. In some instances, well-known structures and components are shown in block diagram form in order to avoid obscuring such concepts.

According to various embodiments, a five actuated degree of freedom (DOF) kinematic mechanism is used that fully automates evaluation of the temporal window quality and can rediscover the temporal window even after complete loss of signal. To those skilled in the art, a distinction exists between an active, or actuated degree of freedom, on the one hand, and a passive degree of freedom on the other hand. An active, or actuated degree of freedom includes an actuator, such as for example, a motor. A passive degree of freedom does not require such an actuator. In this specification, if the term “degree of freedom” is used without being qualified as passive, the degree of freedom discussed is meant to be an active or actuated degree of freedom. In some embodiments, a computer generates commands and directs the mechanism to translate and reorient the probe along the surface of the head until a candidate signal is located. Once located, the probe is reoriented to increase signal strength. In some embodiments, reducing the search time of the automated system to discover the temporal window is accomplished by aligning the mechanism and probe at a known anatomical feature, such as the zygomatic arch. In some embodiments, the alignment is performed with a visual window guide for the user to place the probe at an initial starting point along the zygomatic arch between ear and the eye.

In some embodiments, after the probe is properly aligned, the stiffness of the probe is held normal to the surface at a high enough level to keep the probe seated, but low enough so to be comfortable to the user as the probe moves in and out following the surface of the head. In some embodiments, the X and Y axes can retain a higher servo stiffness in order to maintain precision control of probe location. In some embodiments, because the normal force of the probe is determined by the Z-axis stiffness, the sliding force encounter by the X and Y axes will be limited to a comfortable level, and the probe can be directed to perform a search for the TCD window. In some embodiments, if the orientation of the probe needs to be changed, the orientation stiffnesses can be increased via software.

In some embodiments, the kinematic mechanism of the probe includes five motor, or actuated, degrees of freedom, Q={J1, J2, J3, J4, J5) (i.e., motor or joint space) to effect five degrees of freedom in position and orientation X={x, y, z, pan, tilt} (i.e., task space). As such, the forward kinematics may be written as the relationship between motor coordinates and probe coordinates: X=fwd_kin(Q), where fwd_kin is a function representing a series of equations based on the mechanism design and typically analyzed by Denavit-Hartenberg parameters.

In some embodiments, placement of the TCD probe is specified via the inverse kinematics with either an analytic inverse solution: Q=inv_kin(X), or by using a numerical differential such as the Jacobian inverse solution dQ_{emd}(n)=J⁻¹ (Xₑᵣᵣ(n)), where J is the Jacobian, relating differential motion of the motors to the differential motion of the probe, Xₑᵣᵣ(n) is the probe position and orientation error at time n, and dQ_{emd}(n) is the differential motor command at time n. For mechanisms with more motor or actuated degrees of freedom than probe positon and orientation coordinates being controlled, the kinematics are called redundant, and such mechanisms have more than five motors. For redundant mechanisms, the inverse kinematics changes from the inverse Jacobian to the Moore-Penrose pseudo-inverse (or other generalized inverse) of the Jacobian, dQ_{emd}(n)=J^{†} (Xₑᵣᵣ(n)).

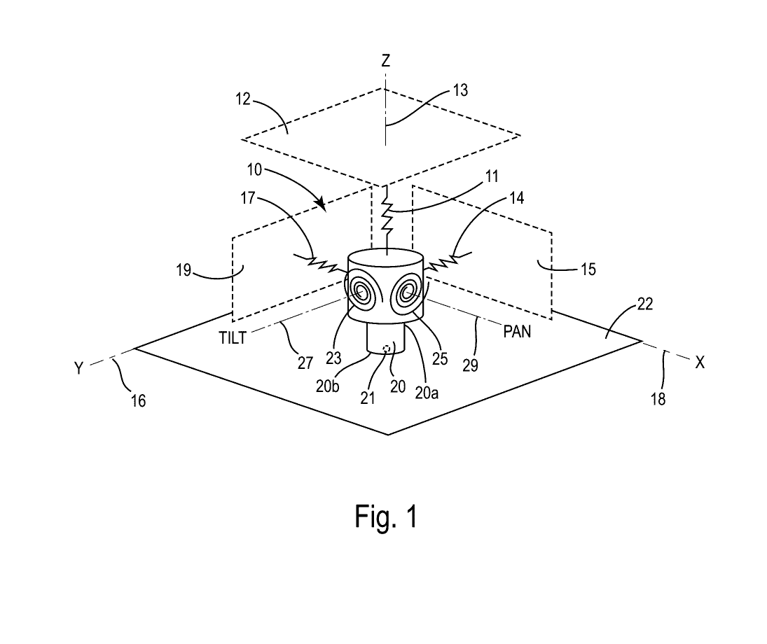

FIG. 1 is a diagram of a model of a virtual support structure 10 for a probe 20, according to an exemplary embodiment. The support structure 10 is configured to position the probe 20 relative to a target surface 22. In some embodiments, the probe 20 is a medical probe, such as a medical probe for use with a transcranial Doppler (TCD) apparatus to emit ultrasound wave emissions directed to the target surface 22. In other embodiments, the probe 20 is configured to emit other types of waves during operation, such as, but not limited to, infrared waves, x-rays, and so on. In various embodiments, the probe 20 may be a transcranial color-coded sonography (TCCS) probe, or it may be an array such as a sequential array or phased array which emits waves.

In some embodiments, the probe 20 has a first end 20a and a second end 20b. In some embodiments, the first end 20a interfaces with the support structure 10. In some embodiments, the second end 20b contacts the target surface 22 on which the probe 20 operates at a contact point 21. In some embodiments, the second end 20b is a concave structure such that the contact point 21 is a ring shape (i.e., the second end 20b contacts the target surface 22 along a circular outer edge of the concave second end 20b). The support structure 10 controls the relative position of the probe 20 (e.g., z-axis force, y-axis force, x-axis force, normal alignment, etc.). The support structure 10 is shown as a virtual structure including a first virtual spring 11 coupled between the probe 20 and a virtual surface 12 and exerting a force along a z-axis 13, a second virtual spring 14 coupled between the probe 20 and a virtual surface 15 and exerting a force along a y-axis 16, and a third virtual spring 17 coupled between the probe 20 and a virtual surface 19 and exerting a force along the x-axis 18. The virtual support structure 10 further includes a torsional spring 23 exerting a torque about a tilt axis 27 and a second torsional spring 25 exerting a torque about a pan axis 29. In some embodiments, the virtual support structure 10 includes other virtual elements, such as virtual dampers (not shown). Virtual dampers represent elements that improve the stability of the system and are useful for tuning the dynamic response of the system. The virtual, or apparent inertia of the probe, can also be set to have isotropic or anisotropic properties, by modeling and feed forwarding out the effects of mechanism inertia, motor rotational inertial, centripetal/centrifugal effects, and replacing them with arbitrary inertial properties, within the physical performance limits of the device.

The virtual support structure 10 represents a variety of mechanical structures that may be utilized to position the probe 20 relative to the target surface 22, as described in more detail below. In some embodiments, the second end 20b of the probe 20 is caused to contact a relatively delicate surface, such as the skin of the patient or subject. The support structure is configured to adjust its stiffness (e.g., impedance, compliance, etc.) to provide variable linear forces and rotational forces on the probe 20, and may be relatively stiff in some directions and may be relatively compliant in other directions. For example, the support structure 10 may apply minimal force and may be relatively compliant along the z-axis 13 to minimize forces applied to the patient or subject (e.g., if the patient or subject moves relative to the support structure) in a direction generally normal to the target surface 22 and may be relatively stiff along the y-axis 16 and the x-axis 18 to improve the positional accuracy and precision of the probe 20 along a plane generally parallel to the target surface 22. Further, the desired stiffness of the support structure 10 along various axes may vary over time, depending on the task at hand. For example, the support structure may be configured to be relatively compliant in scenarios in which the support structure 10 is being moved relative to the patient or subject (e.g., during initial set-up of the probe structure, removal of the probe structure, etc.), or when it is advantageous to be relatively free-moving (e.g., during maintenance/cleaning, etc.), and may be configured to be relatively stiff, in some directions, in scenarios in which accuracy and precision of the positioning of the probe 20 is advantageous (e.g., during the TCD procedure or other procedure being performed with the probe 20).

As described in more detail below, a kinematic model of the support structure 10 can be utilized to calculate the relationship between the forces applied to the target surface 22 by the probe 20 and the forces (e.g., torques) applied by actuators actuating the support structure 10. The forces applied to the target surface 22 by the probe 20 in the idealized system can therefore be determined theoretically, without direct force sensing, thereby eliminating the need for a load cell disposed in-line with the probe 20 and/or a force torque sensor coupled to the probe 20 to maintain appropriate contact force that maximizes signal quality. In a physical system, static friction, along with other unmodeled physical effects, may introduce some uncertainty.

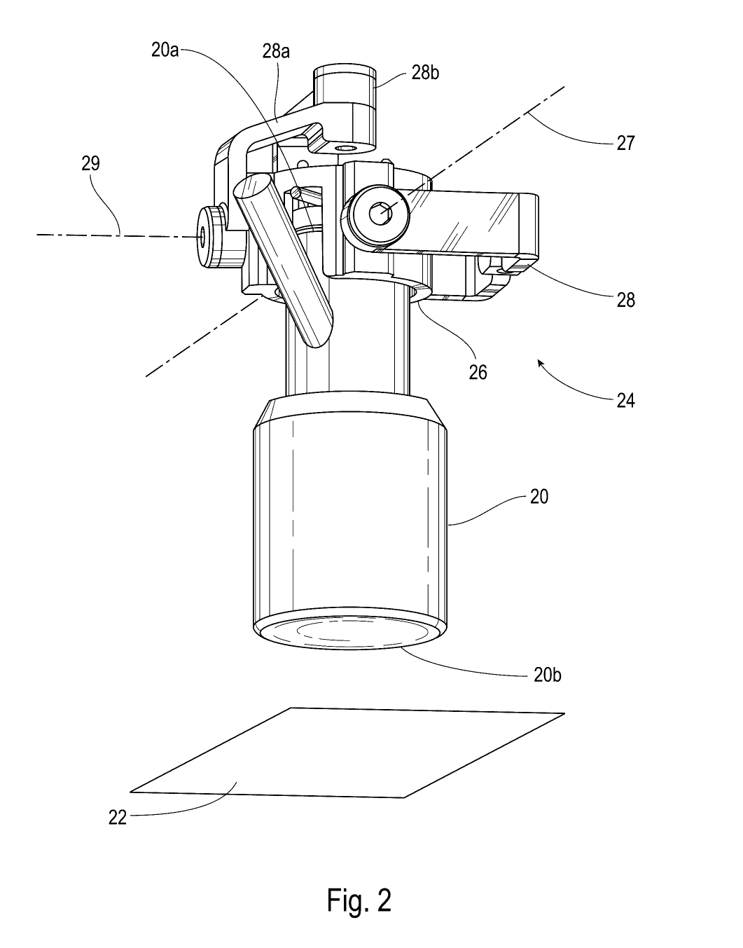

Referring to FIG. 2, the probe 20 is shown according to an exemplary embodiment mounted to a portion of a support structure, shown as a gimbal structure 24, which can rotate about multiple axes, at the first end 20a. The gimbal structure 24 includes a first frame member 26 that is able to rotate about the tilt axis 27 and a second frame member 28 that is able to rotate about the pan axis 29. The target surface 22 may be uneven (e.g., non-planar). The gimbal structure 24 allows the probe 20 to be oriented such that it is normal to the target surface 22 at the contact point 21.

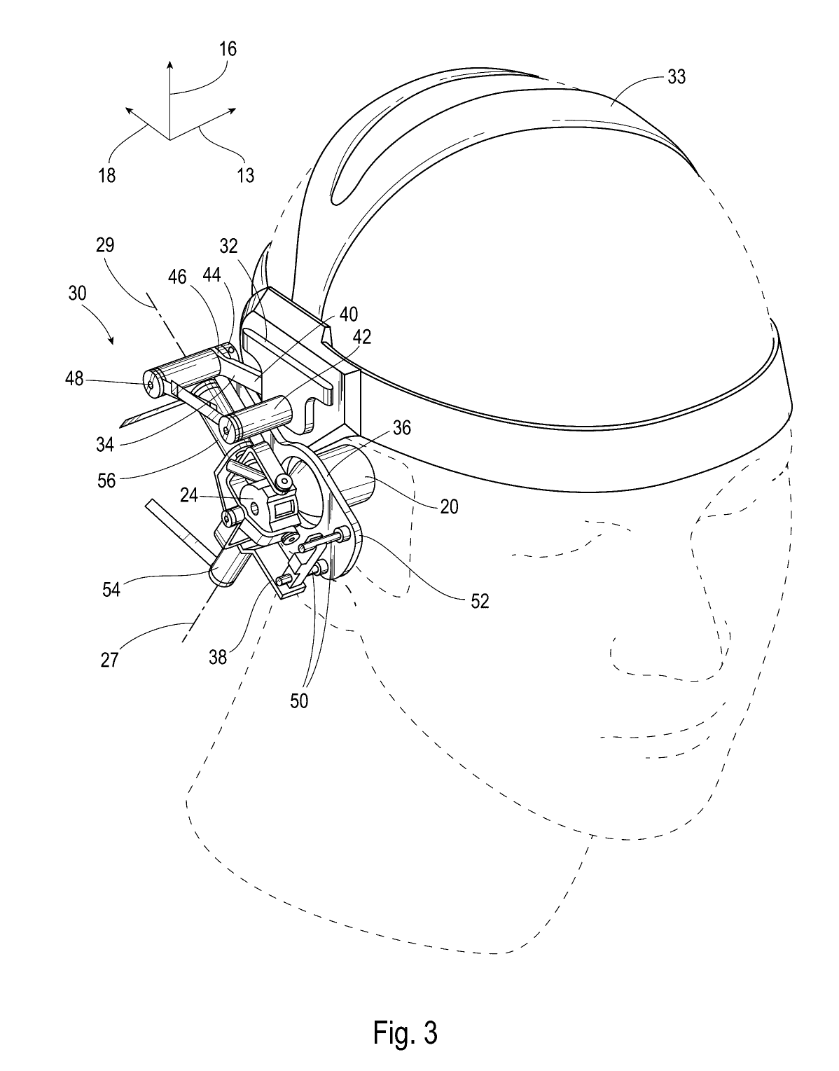

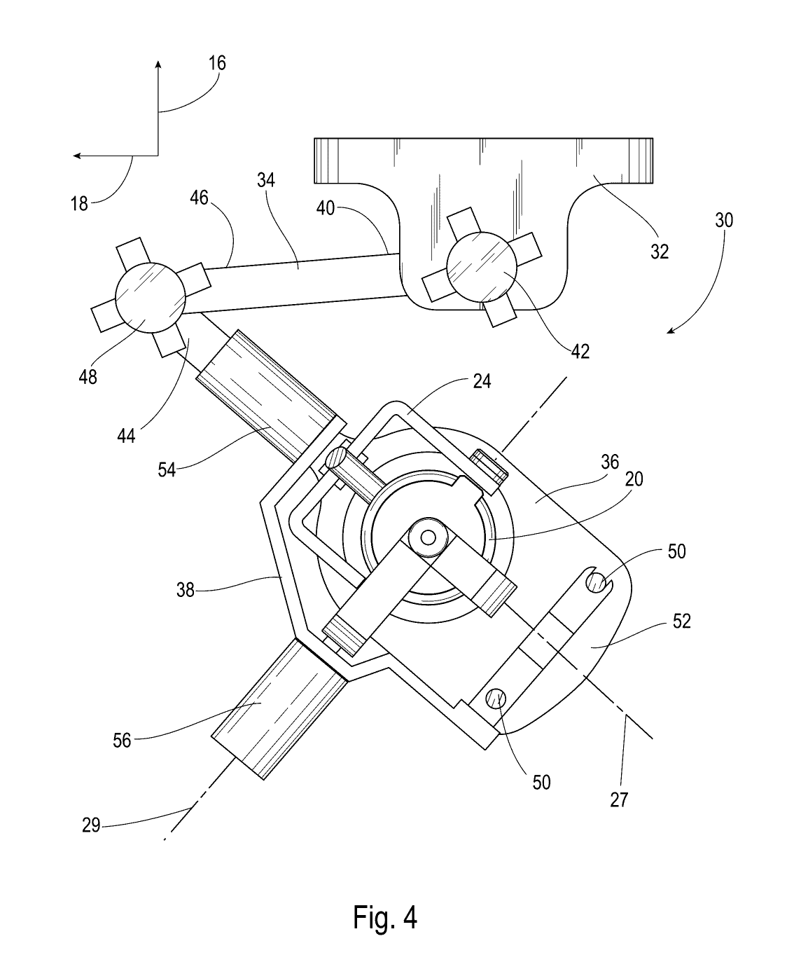

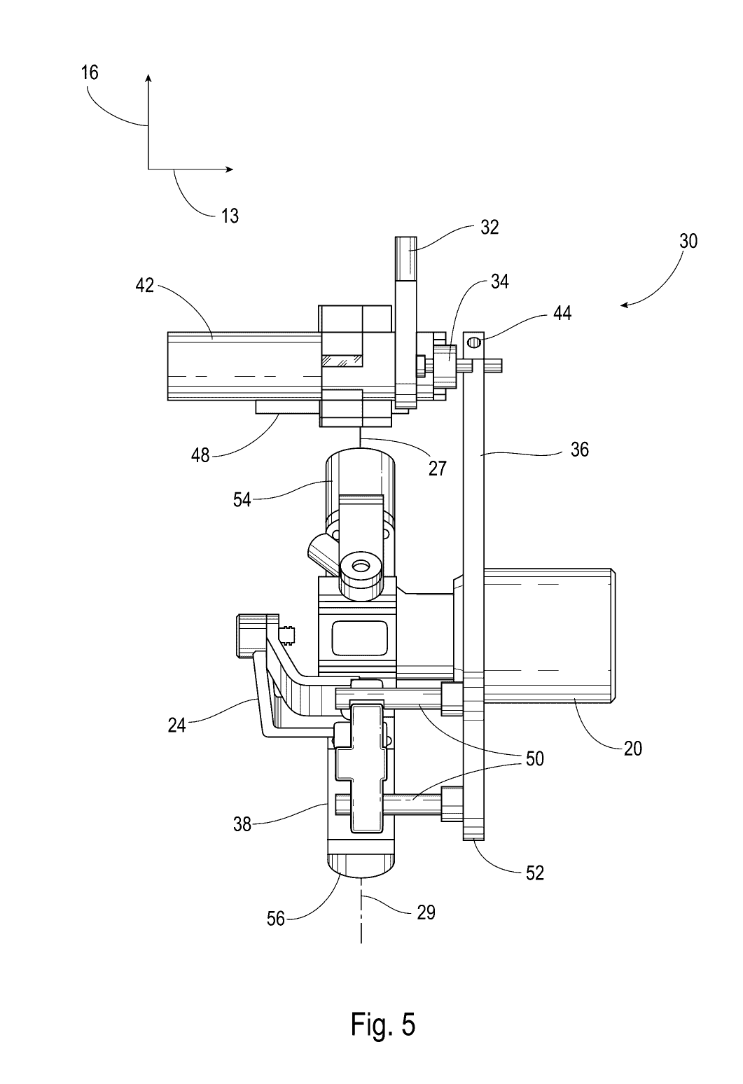

Referring now to FIGS. 3-5, a support structure 30 for the probe 20 is shown according to an exemplary embodiment as a two-link revolute (e.g., revolute-revolute) robot. The support structure 30 includes a first frame member 32, a second frame member 34, a third frame member 36, a fourth frame member 38, and the gimbal structure 24. The first frame member 32 is configured to be a static member. The first frame member 32 may, for example, be mounted to a halo or headset 33 worn on the patient's or subject's head or other structure that attaches the first frame member 32 to the patient or subject or fixes the position of the first frame member 32 relative to the patient or subject. The probe 20 is configured to emit energy into the head of the patient or subject.

Referring to FIG. 3, the second frame member 34 is a link configured to rotate about the z-axis 13. The z-axis 13 is generally perpendicular to the surface of the head. A first end 40 of the second frame member 34 is coupled to the first frame member 32. According to an exemplary embodiment, the rotation of the second frame member 34 relative to the first frame member 32 is controlled by an actuator 42, shown as an electric motor and gearbox that is attached through the first frame member 32. Actuator 42 acts as a perpendicular translation actuator for translating the probe along a perpendicular axis generally perpendicular to the surface of the head.

The third frame member 36 is a link configured to rotate about the z-axis 13. A first end 44 of the third frame member 36 is coupled to a second end 46 of the second frame member 34. According to an exemplary embodiment, the rotation of the third frame member 36 relative to the second frame member 34 is controlled by an actuator 48, shown as an electric motor and gearbox that is attached through the second frame member 34.

The fourth frame member 38 is configured to translate along the z-axis 13 (e.g., in and out, in and away from the head, etc.). According to an exemplary embodiment, the fourth frame member 38 slides along rail members 50 that are fixed to a second end 52 of the third frame member 36. The position of the fourth frame member 38 relative to the third frame member 36 is controlled by an actuator, such as an electric motor and a lead screw (not shown for clarity).

The gimbal structure 24 and the probe 20 are mounted to the fourth frame member 38. The gimbal structure 24 controls the orientation of the probe 20 about the tilt axis 27 and the pan axis 29 (e.g., pan and tilt). The position of the probe 20 about the tilt axis 27 is controlled by an actuator 54, shown as an electric motor and gearbox. Actuator 54 acts as a rotation actuator to rotate the probe. The position of the probe 20 about the pan axis 29 is controlled by an actuator 56, shown as an electric motor and gearbox. Actuator 56 acts as a rotation actuator to rotate the probe. In one embodiment, the rotation of the probe 20 about the tilt axis 27 and the pan axis 29 is different than the z-axis 13, regardless of the rotation of the frame members 34 and 36.

The probe 20 is able to move on the x-y plane, i.e., the translation plane, which is defined by the x-axis 18 and the y-axis 16, through the rotation of the second frame member 34 and the third frame member 36. The probe 20 is able to move along the z-axis 13, i.e., the translation axis, through the translation of the fourth frame member 38. Further, the probe 20 is able to rotate about tilt axis 27 and the pan axis 29 through the gimbal structure 24. Combining these five actuated degrees of freedom allows the position and orientation of the probe 20 relative to the target surface 22 to be completely described and controlled, discounting rotation about a third axis that is orthogonal to the pan axis 29 and the tilt axis 27.

According to an exemplary embodiment, the actuators utilized to position the support structure 30 are servo motors. The use of servo motors to control the support structure allow for a more precise control, compared to a stepper motor, for the torque output, rotational position, and angular speed of the motor, as well as the corresponding position of the probe 20 and the interaction between the probe 20 and the target surface 22. Of course, other suitable motors known to those of ordinary skill in the art could also be used.

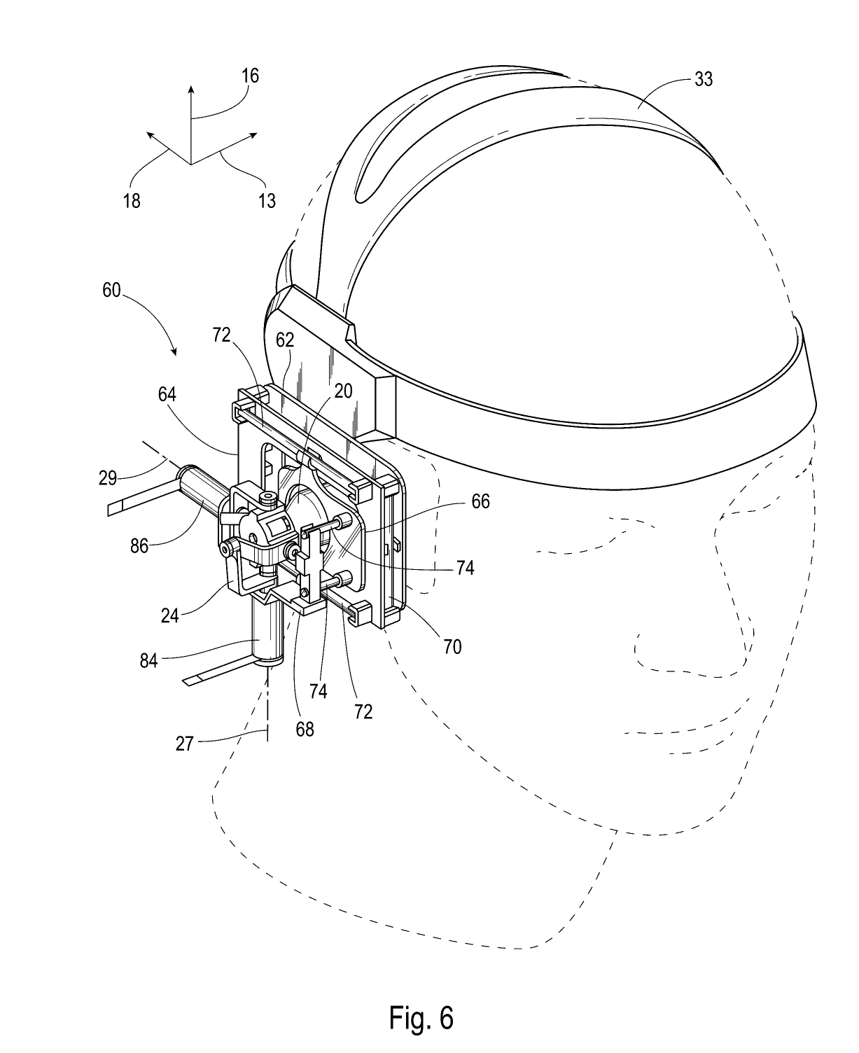

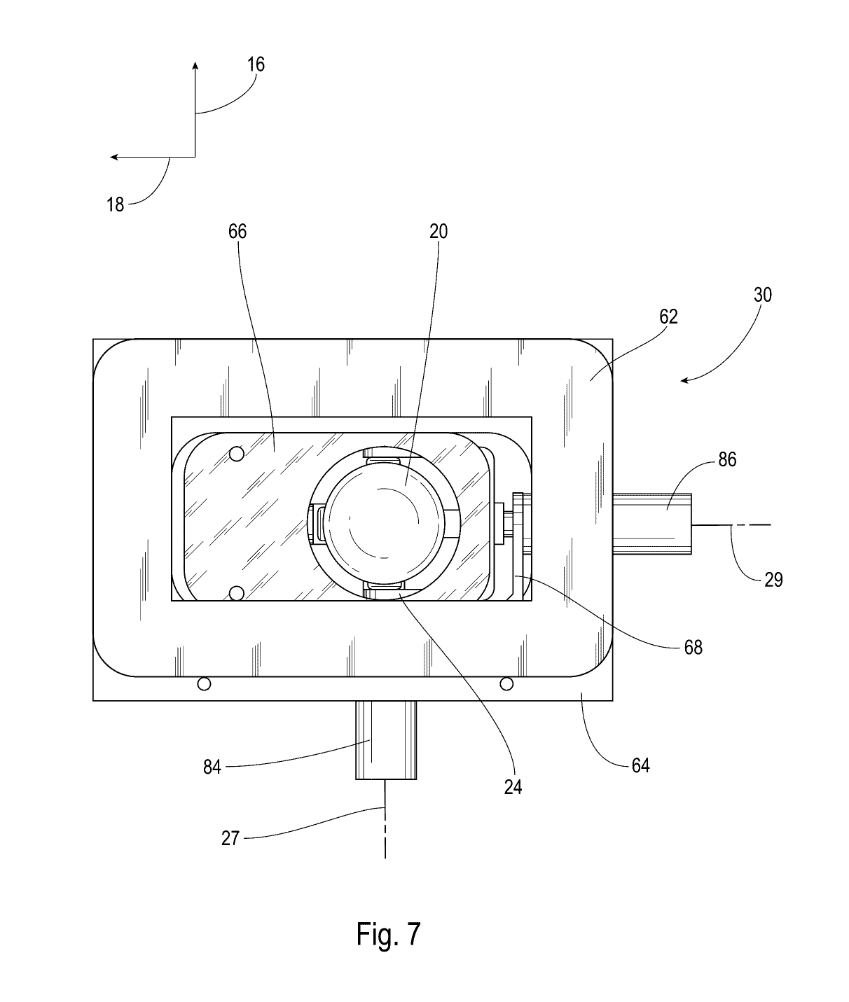

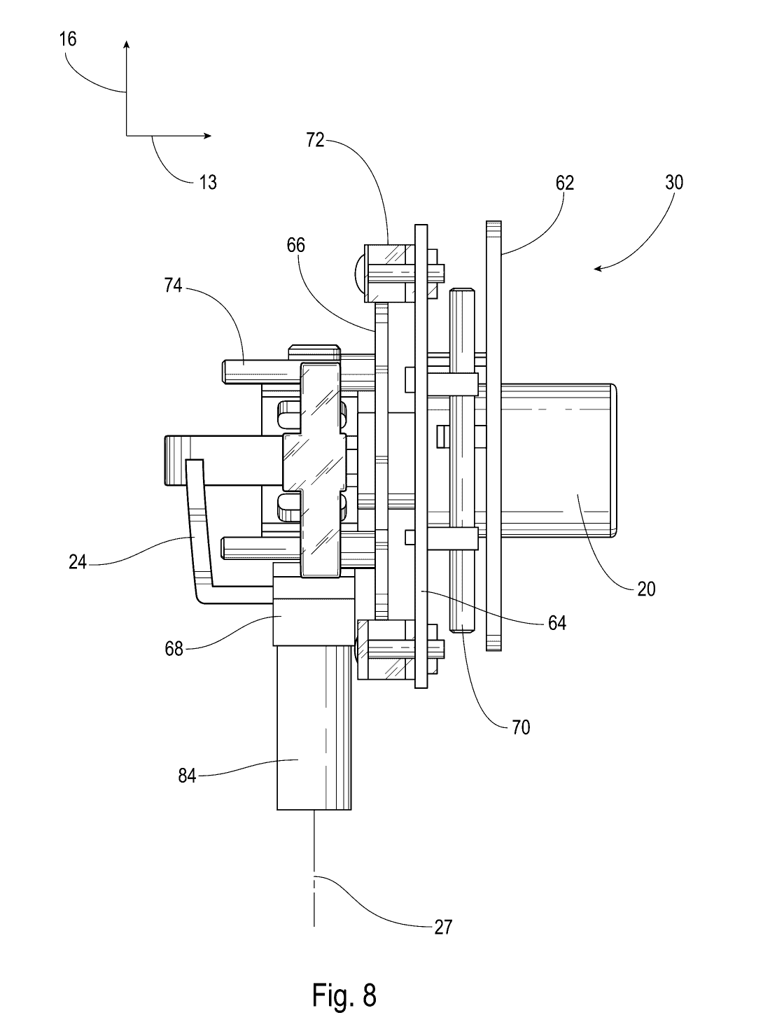

Referring now to FIGS. 6-8, a support structure 60 for the probe 20 and the gimbal structure 24 is shown according to another exemplary embodiment as a prismatic (e.g., Cartesian, rectilinear, etc.) robot. FIG. 6 illustrates an exemplary Prismatic-Prismatic-Prismatic robot. The support structure 60 includes a first frame member 62, a second frame member 64, a third frame member 66, a fourth frame member 68, and the gimbal structure 24. The first frame member 62 is configured to be a static member. The first frame member 62 may, for example, be mounted to a halo or headset 33 worn on the patient's or subject's head or other structure that fixes the position of the first frame member 62 relative to the patient or subject.

The second frame member 64 is configured to translate along the y-axis 16 (e.g., up and down, bottom of ear to top of ear, etc). According to an exemplary embodiment, the second frame member 64 slides along rail members 70 that are fixed to the first frame member 62. The position of the second frame member 64 relative to the first frame member 62 is controlled by an actuator, such as an electric motor and a lead screw (not shown for clarity).

The third frame member 66 is configured to translate along the x-axis 18 (e.g., forward and backward, ear to eye, etc.). According to an exemplary embodiment, the third frame member 66 slides along rail members 72 that are fixed to the second frame member 64. The rail members 72 are orthogonal to the rail members 70. The position of the third frame member 66 relative to the second frame member 64 is controlled by an actuator, such as an electric motor and a lead screw (not shown for clarity).

The fourth frame member 68 is configured to translate along the z-axis 13 (e.g., in and out, in and away from the head, etc.). According to an exemplary embodiment, the fourth frame member 68 slides along rail members 74 that are fixed to the third frame member 66. The position of the fourth frame member 68 relative to the third frame member 66 is controlled by an actuator, such as an electric motor and a lead screw (not shown for clarity).

The gimbal structure 24 and the probe 20 are mounted to the fourth frame member 68. The gimbal structure 24 controls the orientation of the probe 20 about the tilt axis 27 and the pan axis 29 (e.g., tilt and pan). The position of the probe 20 about the tilt axis 27 is controlled by an actuator 84, shown as an electric motor and gearbox. The position of the probe 20 about the pan axis 29 is controlled by an actuator 86, shown as an electric motor and gearbox.

The probe 20 is able to move on the x-y plane through the translation of the second frame member 64 and the third frame member 66, move along the z-axis 13 through the translation of the fourth frame member 68, and rotate about tilt axis 27 and the pan axis 29 through the gimbal structure 24. Combining these five actuated degrees of freedom allows the position and orientation of the probe 20 relative to the target surface 22 to be completely described and controlled, discounting rotation about a third axis that is orthogonal to the pan axis 29 and the tilt axis 27.

A kinematic model can be developed for any embodiment of a support structure for the probe 20 to determine the relationship between the forces exerted at the probe 20 and the forces applied by the actuators controlling the support structure.

A stiffness matrix for the support structure is first determined. The stiffness matrix is determined using a multitude of variables, including the physical properties of the support structure (e.g., the geometry of the frame members, the stiffness of the individual frame members etc.), the system stiffness along the chosen coordinate system axis, and a velocity-based term for system damping. According to an exemplary embodiment, the desired stiffness of the support structure is defined in the z direction (K_{z}), the y direction (K_{y}), and the x direction (Kₓ)(e.g., as represented by the virtual springs 11, 14, and 17 in FIG. 1), and about the pan axis 29 (Kωₓ) and about the tilt axis 27 (Kω_{y})(e.g., as represented by the virtual torsional springs 23 and 25 in FIG. 1). As described above, in some embodiments, the virtual stiffnesses vary over time and are based on the task being accomplished with the probe 20. For example, stiffness in the y direction and in the x direction may have a lower bound corresponding to a relatively low lateral stiffness during a set-up or removal procedure, in which the support structure is configured to be relatively compliant; and an upper bound corresponding to a relatively high stiffness during a scanning procedure, in which the support structure is configured to be relatively stiff, allowing for a more accurate positioning of the probe 20. Likewise, stiffness in the z direction may have a lower bound corresponding to a relatively low stiffness during initial positioning of the probe 20 in the z direction, in which the support structure is configured to be relatively compliant to allow the probe 20 to self-align (e.g., to minimize discomfort for the patient or subject); and an upper bound corresponding to a relatively high stiffness during a scanning procedure, in which the support structure is configured to more stiff, to overcome friction forces between the probe 20 and the target surface 22 and to maintain the orientation of the probe 20. Further, rotational stiffnesses about the y axis and the x axis may have a lower bound corresponding to a relatively low rotational stiffness during positioning of the probe 20 to conform to the contour of the target surface 22 (e.g., the head of the patient or subject), in which the support structure (e.g., the gimbal structure 24) is configured to be relatively compliant (e.g., to minimize discomfort for the patient or subject); and an upper bound corresponding to a relatively high rotational stiffness when a more accurate positioning (e.g., panning, tilting, etc.) of the probe 20 is desired.

A force vector is then derived using the following equation:

\vec{F}=KΔ\vec{x} (Eq. 1)

where K is the stiffness matrix and Δ\vec{x} is the vector of the difference of the desired and actual translational position in the x, y, and z directions and rotational position about the x-axis 18 and y-axis 16 of the probe 20.

The force applied by the actuators (e.g., the torque applied by rotational actuators) controlling the position of the support structure may then be determined using the following equation:

τ=J^{T}\vec{F} (Eq. 2)

where J^{T} is the Jacobian transpose determined by the kinematics of the specific support structure. The Jacobian is the differential relationship between the joint positions and the end-effector position and orientation (e.g., the position of the probe 20). The joint positions are either in units of radians (e.g., for rotational joints), or in units of length (e.g., for prismatic or linear joints). The Jacobian is not static and changes as the support structure position articulates.

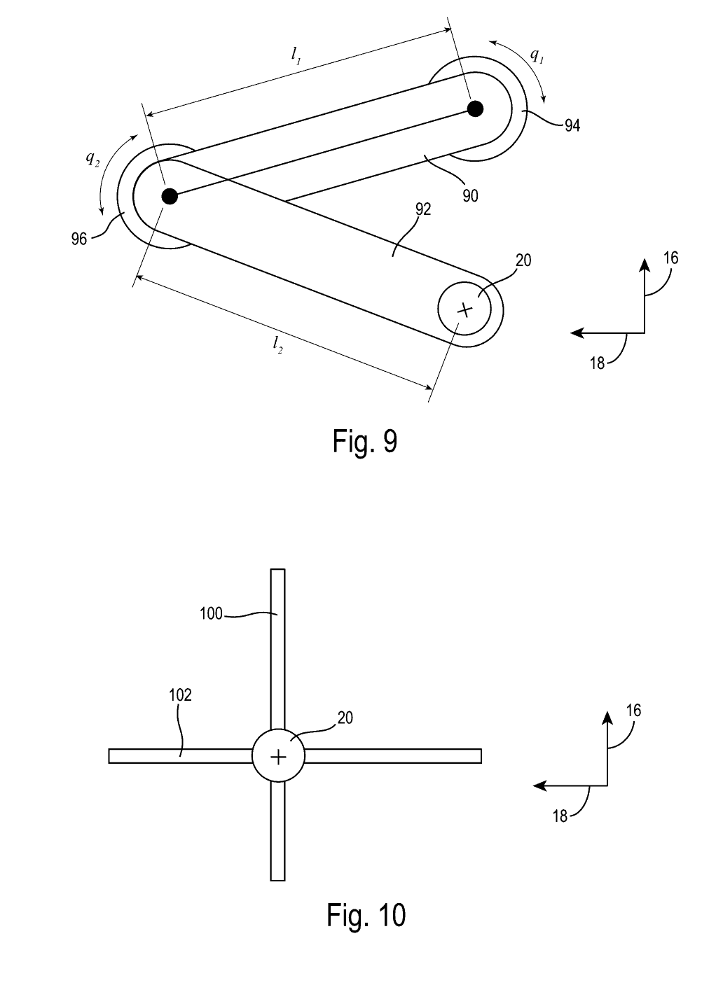

Referring now to FIG. 9, a schematic front view diagram of the support structure 30 is shown. The second frame member 34 is represented by a first link 90, having a length l₁. The first link 90 is articulated by a rotary actuator 94, the rotation of which is shown as q₁. The third frame member 36 is represented by a second link 92, having a length l₂. The second link 92 is articulated by a rotary actuator 96, the rotation of which is shown as q₂. The actuators 94 and 96 move the probe 20 in the x-y plane.

The forward kinematics of this device are:

c₁=cos(q₁), s₁=sin(q₁)

c₁₂=cos(q₁+q₂), s₁₂=sin(q₁+q₂)

x=l₁c₁+l₂c₁₂ (Eq. 3)

y=l₁s₁+l₂s₁₂ (Eq. 4)

The Jacobian for such a revolute-revolute robot is derived by taking the partial derivative of the forward kinematics with respect to both q₁ and q₂.

$$J = [ \begin{matrix}- {l}_{1} {s}_{1} - {l}_{2} {s}_{12} & - {l}_{2} {s}_{12} \\ {l}_{1} {c}_{1} + {l}_{2} {c}_{12} & {l}_{2} {c}_{12}\end{matrix} ] \qquad ( Eq . 5 )$$

The Jacobian shown in Equation 5 is the Jacobian for the Cartesian movement of the revolute-revolute robot on the x-y plane (e.g., translation along the y-axis 16 and the x-axis 18), describing the differential relationship between joint motion and probe motion. One of ordinary skill in the art would understand that in other embodiments, additional terms may be included in the Jacobian to describe the differential relationship between the motion of the probe 20 and other motions of the robot (e.g., rotation of the probe 20 about the tilt axis 27 and the pan axis 29 and translation along the z-axis 13).

Referring now to FIG. 10, a schematic front view diagram of the support structure 60 is shown. The probe 20 is moved in the y direction by a first linear actuator 100 (e.g., an electric motor and lead screw) and is moved in the x direction by a second linear actuator 102 (e.g., an electric motor and lead screw). The actuators 100 and 102 move the probe 20 in the x-y plane. Because each joint is orthogonal to the other, and has a one to one mapping of joint motion to Cartesian motion, the Jacobian for such a prismatic robot becomes the identity matrix:

$$J = [ \begin{matrix}1 & 0 \\ 0 & 1\end{matrix} ] \qquad ( Eq . 6 )$$

The Jacobian shown in Equation 6 is the Jacobian for the Cartesian movement of the prismatic robot on the x-y plane (e.g., translation along the y-axis 16 and the x-axis 18), describing the differential relationship between joint motion and probe motion. In other embodiments, additional terms may be included in the Jacobian to describe the differential relationship between the motion of the probe 20 and other motions of the robot (e.g., rotation of the probe 20 about the tilt axis 27 and the pan axis 29 and translation along the z-axis 13).

Referring to FIG. 3, The support structure 30 controls the position of the probe 20 in the z direction with the translation of the fourth frame member 38 with a single linear actuator (e.g., an electric motor and lead screw). Referring to FIG. 6, Similarly, the support structure 60 controls the position of the probe 20 in the z direction with the translation of the fourth frame member 68 with a single linear actuator (e.g., an electric motor and lead screw). For either support structure, there is a direct correlation between the position of the actuator and the position of the probe 20.

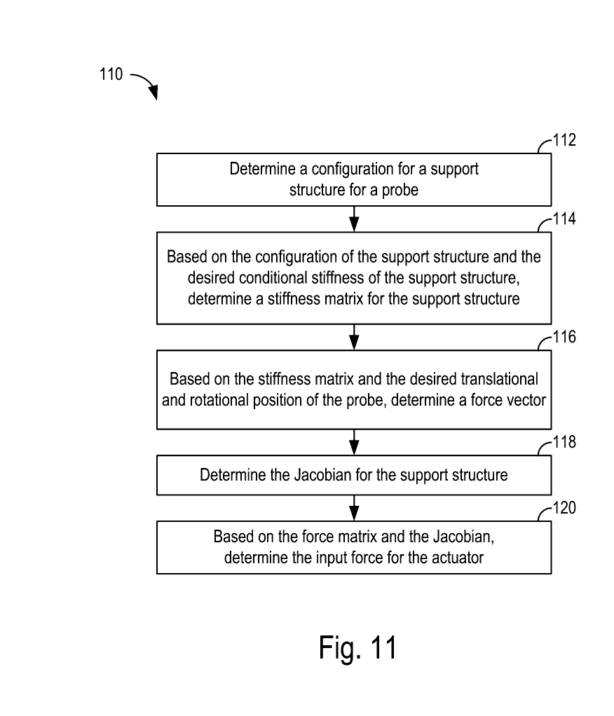

Referring now to FIG. 11, a method 110 of determining the input force, or torque, for an actuator for a probe support structure is shown according to an exemplary embodiment. The configuration of the support structure for a probe is first determined (step 112). The configuration may include any number of revolute joints and/or prismatic joints. In some embodiments, the support structure provides translation of the probe along one or more axis (e.g., the x, y, and z axis in a Cartesian coordinate system; the r, θ, and z axis in a polar coordinate system, etc.) and/or rotation about one or more axis.

Based on the configuration of the support structure and the desired variable stiffness of the support structure, a stiffness matrix for the support structure is determined (step 114). The stiffness matrix includes terms based on the physical properties of the support structure, including the geometry of the frame members and the stiffness of the individual frame members, the desired stiffness of the support structure in the z direction (Kz), the y direction (Ky), and the x direction (Kx), the desired rotational stiffness of the support structure (Kωₓ, Kω_{y}), and a velocity-based term for system damping.

Based on the stiffness matrix and the desired translational and rotational position of the probe, a force vector is determined (step 116). The desired position of the probe may be determined using any coordinate system. According to an exemplary embodiment, the force vector is derived from the product of the stiffness matrix and a matrix of the desired translational and rotational position of the probe, as shown in Equation 1.

The Jacobian for the support structure is then calculated (step 118). The Jacobian is determined by the kinematics of the specific support structure. The Jacobian is the differential relationship between the joint positions and the end-effector position. The joint positions are either in units of radians (e.g., for rotational joints), or in units of length (e.g., for prismatic or linear joints). The Jacobian is not static and changes as the support structure position articulates.

Based on the force vector and the Jacobian, the input force for the actuator is determined (step 120). According to an exemplary embodiment, the input force for the actuator is derived from the product of the Jacobian and the force vector, as shown in Equation 2.

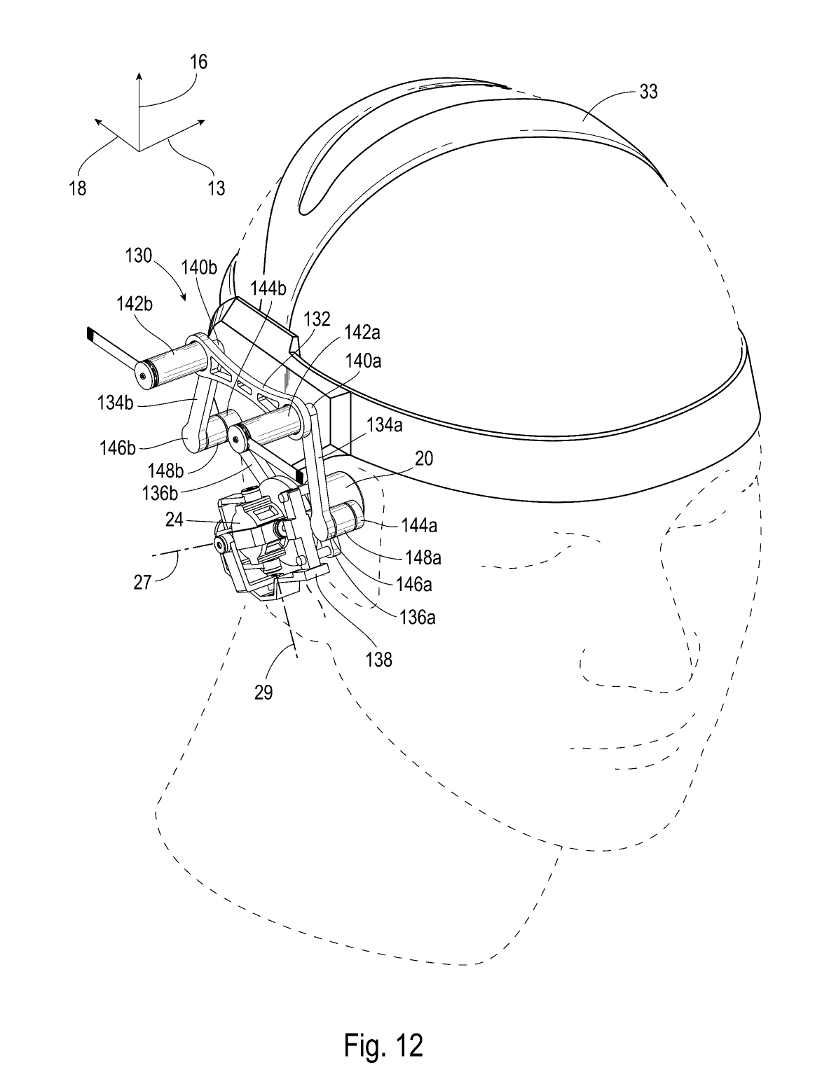

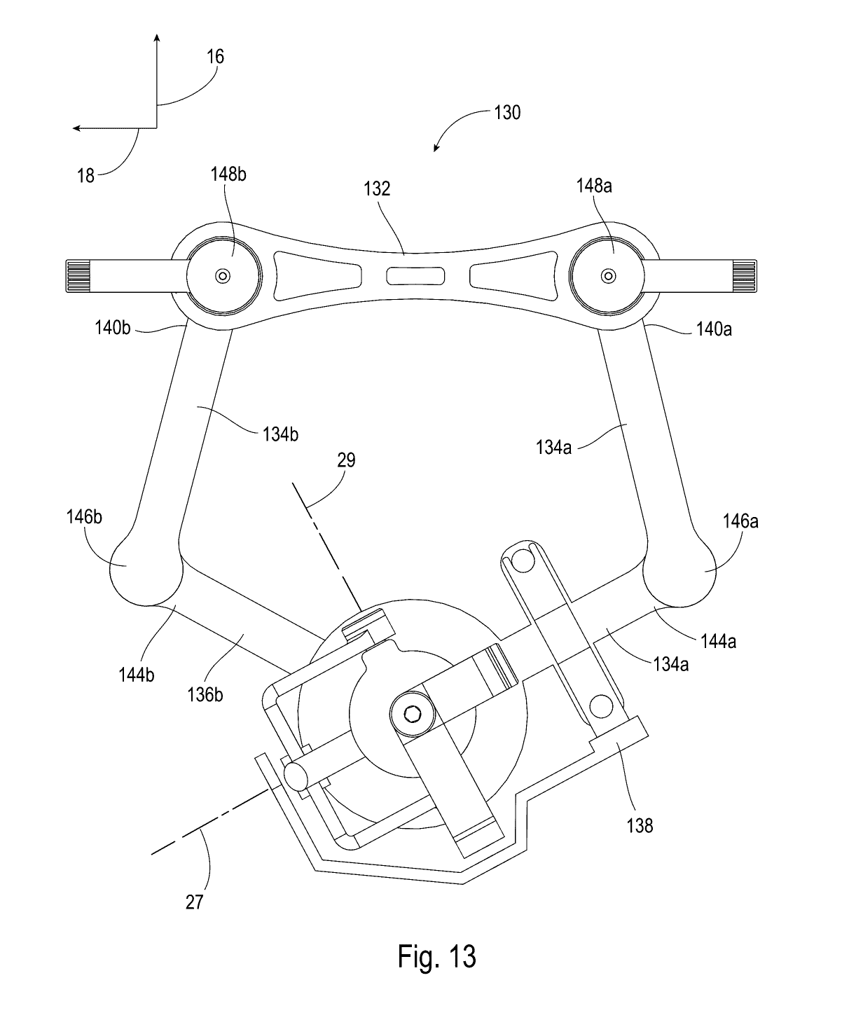

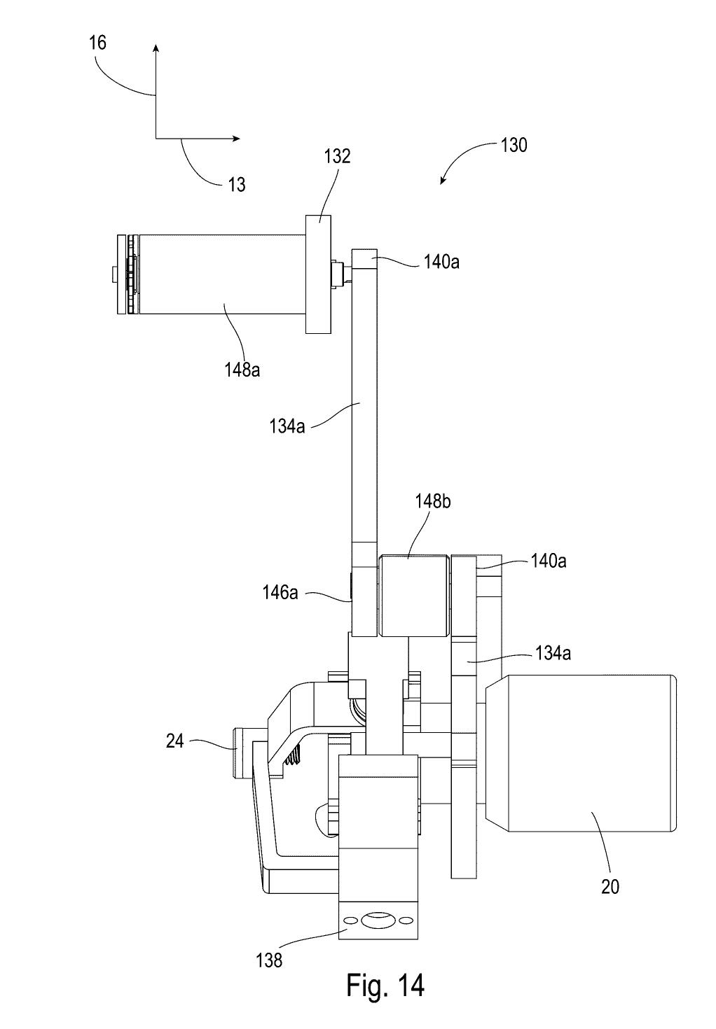

Referring now to FIGS. 12-14, a support structure 130 for the probe 20 is shown according to another exemplary embodiment as a five-link revolute robot. The support structure 130 includes a first frame member 132; a pair of proximal members coupled to the first frame member 132, shown as a second frame member 134a and a third frame member 134b; a pair of distal members coupled to the respective proximal frame members and to each other, shown as a fourth frame member 136a and a fifth frame member 136b; a sixth frame member 138 coupled to the distal frame members; and the gimbal structure 24. The first frame member 132 is configured to be a static member. The first frame member 132 may, for example, be mounted to a halo or headset 33 worn on the patient's or subject's head or other structure that fixes the position of the first frame member 132 relative to the patient or subject.

The second frame member 134a and the third frame member 134b are links configured to rotate about the z-axis 13. A first end 140a of the second frame member 134a is coupled to the first frame member 132. Similarly, a first end 140b of the third frame member 134b is coupled to a separate portion of the first frame member 132. According to an exemplary embodiment, the rotation of the second frame member 134a relative to the first frame member 132 is controlled by an actuator 142a, shown as an electric motor and gearbox that is attached through the first frame member 132. According to an exemplary embodiment, the rotation of the third frame member 134b relative to the first frame member 132 is controlled by an actuator 142b, shown as an electric motor and gearbox that is attached through the first frame member 132.

The fourth frame member 136a and the fifth frame member 136b are links configured to rotate about the z-axis 13. A first end 144a of the fourth frame member 136a and a second end 146a of the second frame member 134a are each coupled to a hub member 148a via bearings (e.g., press fit bearings, etc.). Similarly, a first end 144b of the fifth frame member 136b and a second end 146b of the third frame member 134b are each coupled to a hub member 148b via bearings (e.g., press fit bearings, etc.).

The fourth frame member 136a and the fifth frame member 136b are coupled together via a bearing (e.g., a press fit bearing, etc.) to form a five-bar linkage. The hub members 148a and 148b offset the proximal members from the distal members along the z-axis 13, which allows the proximal frame members (e.g., second frame member 134a and third frame member 134b) to move freely past the distal frame members (e.g., fourth frame member 136a and fifth frame member 136b) as the links are rotated by the actuators 142a and 142b.

The gimbal structure 24 and the probe 20 are mounted to the sixth frame member 138. The sixth frame member 138 is coupled to one of the distal members (e.g., fourth frame member 136a or fifth frame member 136b) and is configured to translate the gimbal structure 24 and the probe 20 along the z-axis 13 (e.g., in and out, in and away from the head, etc.). The sixth frame member 138 may translate, for example, on rails, as described above in regards to the fourth frame member 38 of the support structure 30 (see FIGS. 3-5). The gimbal structure 24 controls the orientation of the probe 20 about the tilt axis 27 and the pan axis 29 (e.g., pan and tilt). The position of the probe 20 about the tilt axis 27 is controlled by an actuator (not shown), such as an electric motor and gearbox. The position of the probe 20 about the pan axis 29 is controlled by an actuator (not shown), such as an electric motor and gearbox. In one embodiment, the rotation of the probe 20 about the tilt axis 27 and the pan axis 29 is different than the z-axis 13, regardless of the rotation of the frame members 134 and 136.

The probe 20 is able to move on the x-y plane through the movement of the five-bar linkage formed by the first frame member 132, the second frame member 134a, the third frame member 134b, the fourth frame member 136a, and the fifth frame member 136b. The probe 20 is able to move along the z-axis 13 through the translation of the sixth frame member 138. Further, the probe 20 is able to rotate about tilt axis 27 and the pan axis 29 through the gimbal structure 24. Combining these five actuated degrees of freedom allows the position and orientation of the probe 20 relative to the target surface 22 (See FIGS. 1-2) to be completely described and controlled, discounting rotation about a third axis that is orthogonal to the pan axis 29 and the tilt axis 27.

According to an exemplary embodiment, the actuators utilized to position the support structure 130 are servo motors. Of course, any suitable motors could be used instead of servo motors. The use of servo motors to control the support structure allow for a more precise control, compared to a stepper motor, over the rotational position and angular speed of the motor, as well as the corresponding position of the probe 20 and the interaction between the probe 20 and the target surface 22.

The input forces for the actuators 142a and 142b can be calculated in a manner similar to that described above by determining the force vector, determining the forward kinematics of the support structure 130, and calculating the Jacobian by taking the partial derivative of the forward kinematics with respect to the rotations of each of the actuators 142a and 142b.

In some embodiments, for probe 20 contact and seating, instead of trying to predict and control the exact position and orientation of the probe 20, the impedance of the probe 20 is selectively controlled, whether by mechanical design or through software. As such, the orientation degrees of freedom of the probe 20 can be compliant so that they rotate against contact and seat the probe 20 flush with the head, while the translation degrees of freedom are stiff enough to move the probe 20 and keep it placed against the head. In some embodiments, each of the directions has different impedances.

In some embodiments, software is implemented to limit motor torque and motor servo stiffness of the probe 20. In some embodiments, there may be different limits for each direction, creating different stiffnesses in different directions. In some embodiments, the pan and tilt are very compliant, while the translational motions are moderately stiffer. In some embodiments, stiffness through the probe 20 is more compliant than the X, Y translational degrees of freedom.

In some embodiments, software is implemented for task space impedance control. In other words, there can be considered the probe 20 orientation to define a local coordinate system with the Z axis through the center of the probe 20. Instead of manipulating the impedance of the probe 20 by adjusting motor servo stiffness and torque limiting, in some embodiments, the kinematics of the entire robot can be considered to set the impedance of each of the five directions, X, Y, Z, pan, and tilt, local to the probe's 20 coordinate frame. As such, the probe 20 can be more compliant through the center line of the probe 20, but still maintain contact with the surface of the skin, but have local X and Y stiffness sufficient to control the location of the probe 20 with precision.

According to various embodiments, the probe 20 includes a series elastic actuator. In some embodiments, the impedance of the device is altered by adding a compliant member into the mechanical design, either as a spring element into the motor or as a structural member of the robot. In some embodiments, measurement of the amount of deflection is implemented in order to measure the exact position and orientation of the probe 20. A series elastic actuator has the benefit of being designed to an exact compliance, and even has a damping element added, while avoiding computational nonlinearities and instabilities associated with programming the impedance.

In some embodiments, the force is indirectly measured by monitoring the applied current of the motor. For the static case, taking into account the kinematics of the robot, the force/torque vector of the system is computed from the Jacobian: F=(J^{T})⁻¹τ, where τ is the vector of motor torques as predicted by the applied current to the motor.

In some embodiments, the interaction force and torque between the probe 20 and the head is controlled by placing a force/torque sensing mechanism behind the probe 20. The position and orientation of the probe is specified in relation to the measured forces and torques to achieve a desired force/torque vector. This type of closed loop controller is called admittance control and is programmed into the software. Admittance control relates the measured force to a desired probe position and orientation, as illustrated in the following equation.

\vec{X}_{desired}=G_{control}\vec{F}_{measured}

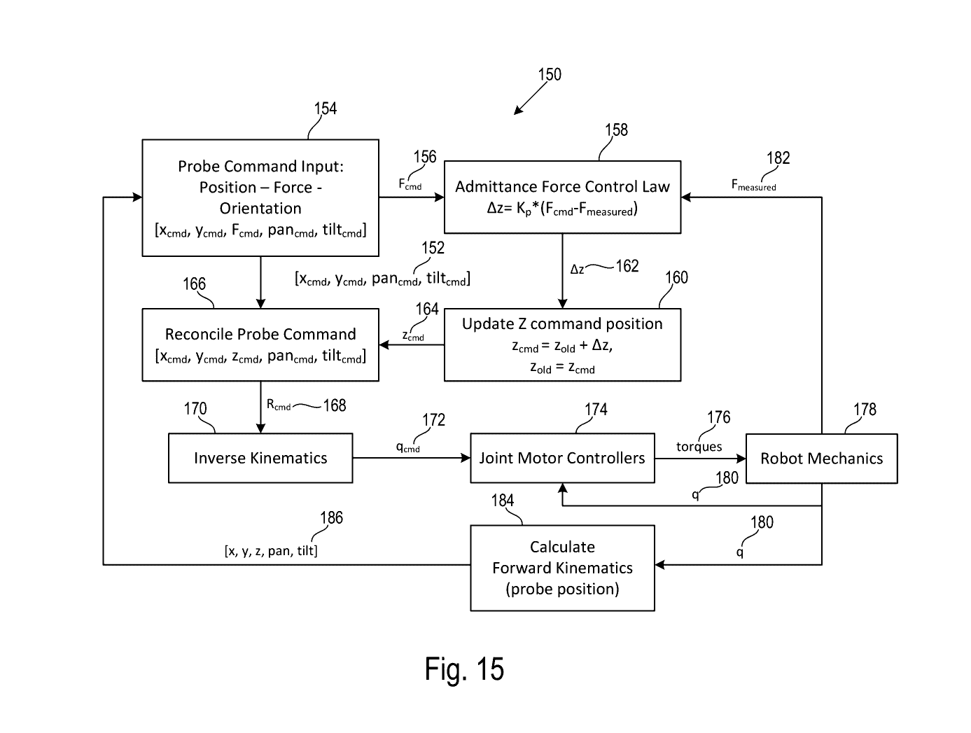

The desired position vector is used to compute desired joint positions from the inverse kinematics. Motor joint controllers are programed with high servo stiffness to enhance disturbance rejection and have low servo tracking errors. A hybrid position-force admittance controller 150 is illustrated in FIG. 15.

In this example of a hybrid position-force controller, force is controlled in the z direction of the probe, while position is controlled in the x and y directions, and the pan and tilt orientations. A hybrid command of positions and force in task space, [x_{cmd},y_{cmd},F_{cmd},pan_{cmd},tilt_{cmd}] 152, is sent by the probe command input block 154, which determines task oriented moves of the probe, such as searching. In this case the positon and orientation is specified in x, y, pan and tilt, and is given in units of length and angle (mm and radians). Force is specified in the z direction, and is given in units of Newtons. For an admittance controller, the force command must be transformed into a desired position. The force command, F_{cmd} 156, is extracted from the probe input command block 154 as used as the command for the admittance force control block 154. Using the measured force, F_{measured}, a change in z position, Δz, is computed by the admittance force control law block 158. A simple, single proportional gain controller is illustrated, but other controller forms can be used. In the update z command position block 160, Δz 162 is added to the old z command position to create an updated z command, Z_{cmd} 164. This information is merged with the other probe position and orientation commands in the reconcile probe command block 166. The reconciled command, R_{cmd} 168 specifies the probe position and orientation in units of length and angle, [x_{cmd},y_{cmd},z_{cmd},lan_{cmd},tilt_{cmd}]. The inverse kinematics block 170 uses the reconciled command, R_{cmd} 168 to solve for the new robot joint command position, q_{cmd} 172, based on the mechanics of the specific robot. It is understood from the discussion above that the robot could have a direct analytic inverse solution, or a numerical solution based on the inverse or pseudo inverse Jacobians depending on the number of degrees of freedom.

The joint command position, g_{cmd} 172, is used as the input for the inner position control loop comprised of the joint motor controllers block 174, the output torques 176, robot mechanics block 178, which is the physical robot, and the measured joint positions, q 180. The robot mechanics block 178 also includes a force sensor which outputs the measured force, F_{measured} 182. The measured force, F_{measured} 182 is the second input to the admittance force control law block 158, which closes the force control loop.

The measured joint positions, q 180, are used as the to the calculate forward kinematics block 184, which computes the current probe positon and orientation [x,y,z,pan,tilt] 186, and sends it back to the probe command input block 154, for use in its probe command generation algorithm.

When combined with an impedance law, different directional and orientational stiffnesses at the probe 20 can be programmed. Admittance control is appropriate for non-backdriveable motors, which are more difficult to use with pure impedance controllers because, without a force-torque sensor, the static friction resisting the user is unobservable while at rest.

Other configurations of the support structure include over and under actuated mechanisms. An over-actuated mechanism, or redundant manipulator, includes more actuated degrees of freedom than task space coordinates that are attempted to be controlled; e.g., the number of motors, Q={J1, J2, J3, J4, J5, . . . ) is greater than the five degrees of freedom in position and orientation X={x, y, z, pan, tilt} of the probe that is being controlled. For such mechanisms there will be many, and possibly an infinite number, of inverse kinematic solutions.

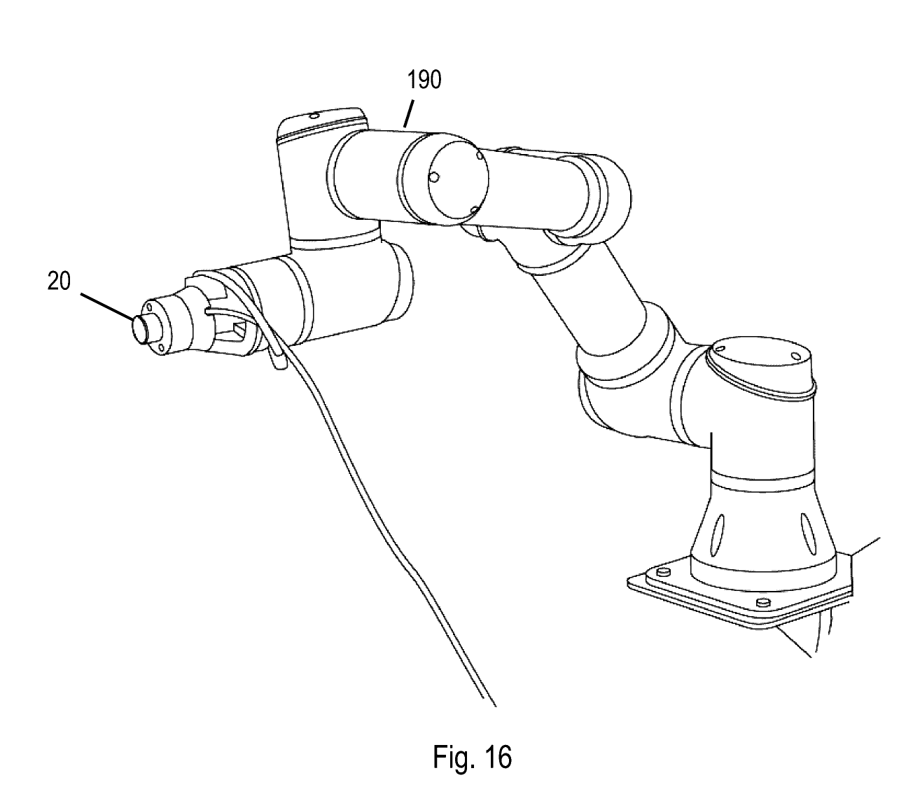

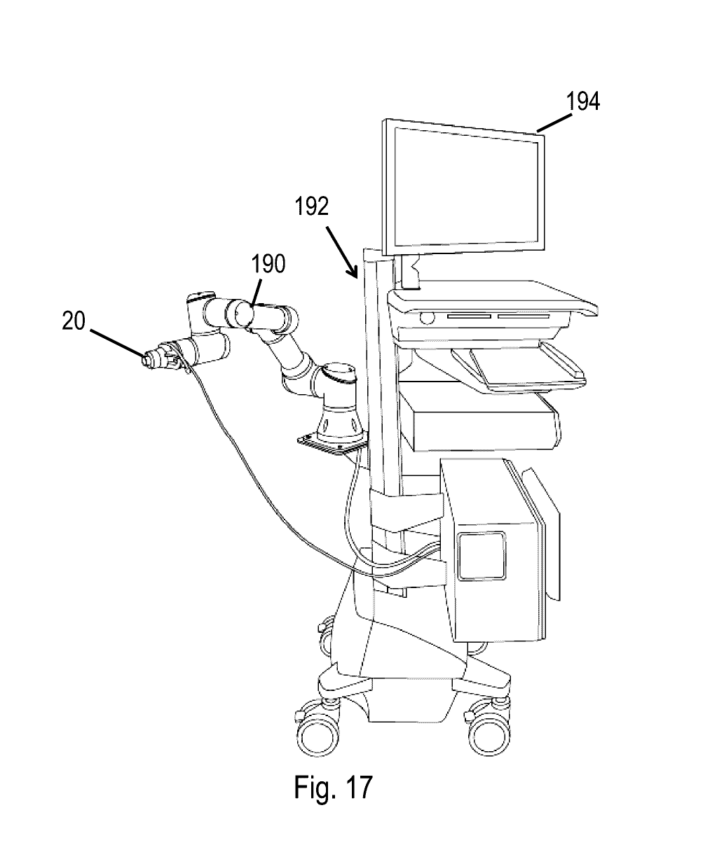

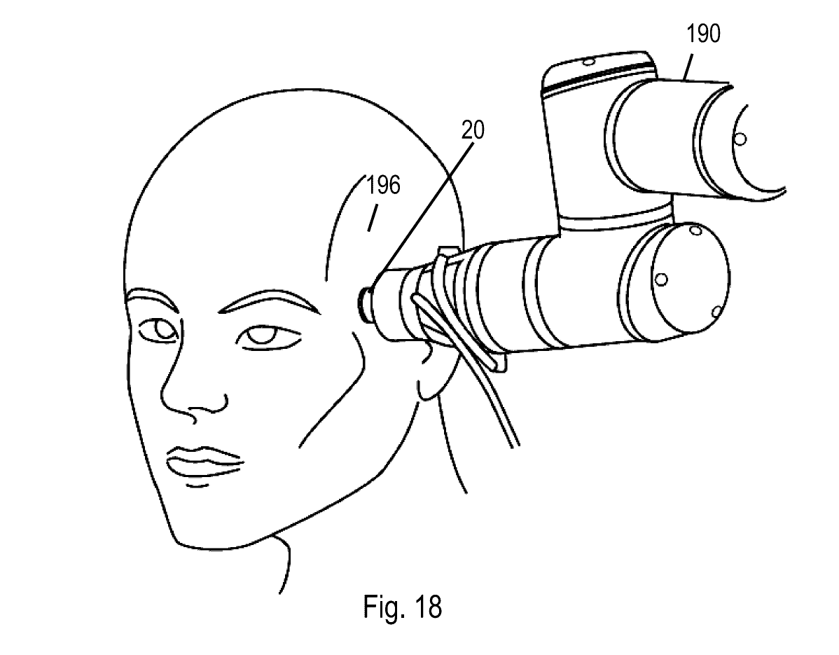

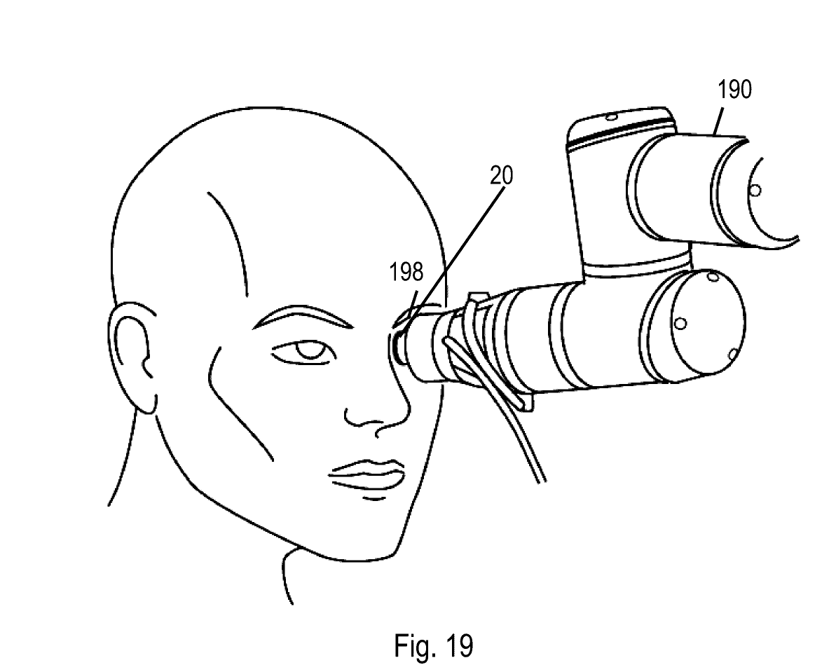

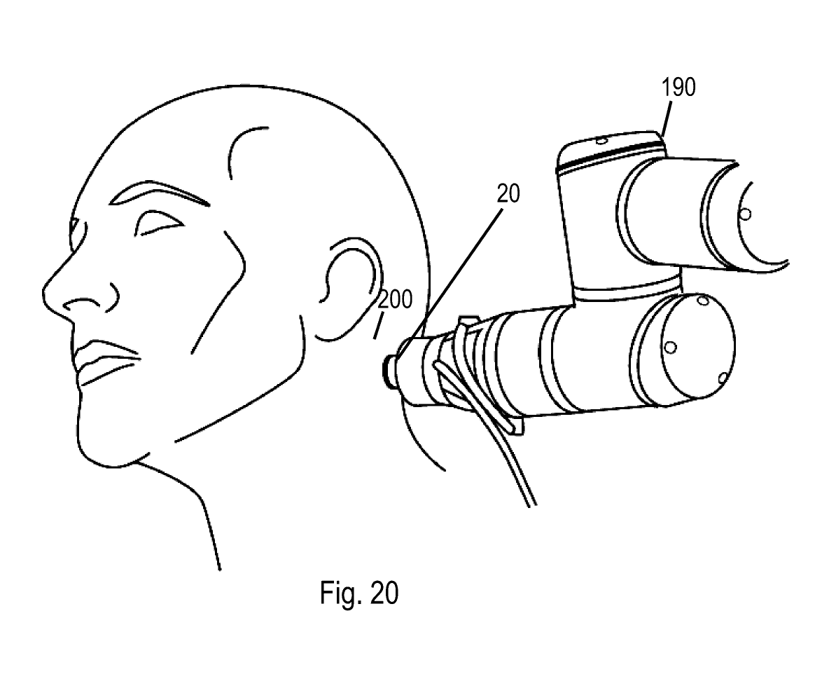

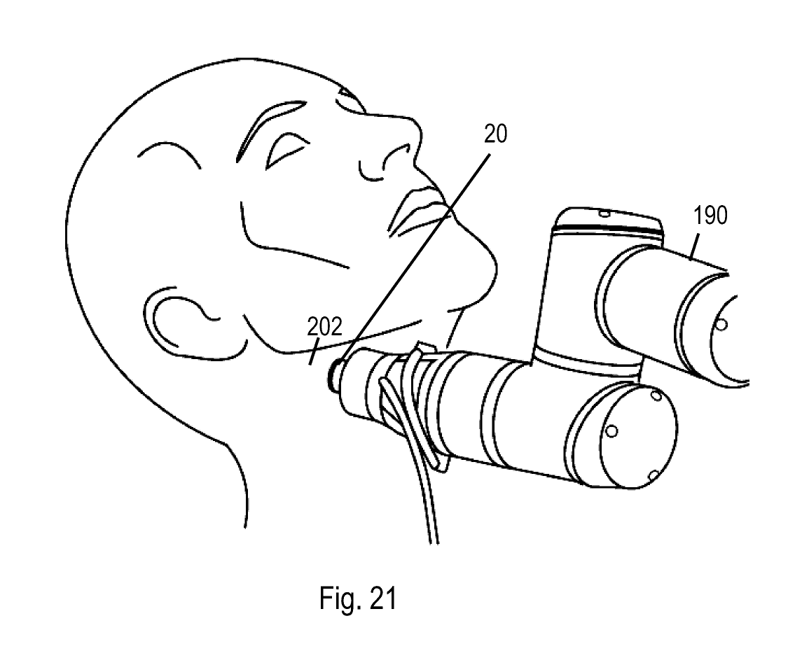

An example of an over-actuated mechanism, or redundant manipulator, is the UR3 Robot, made by Universal Robots, which has six rotating joints, each controlled by its own actuator or motor, to allow for six actuated degree of freedom movement. Referring to FIG. 16, in some embodiments, a probe 20 can be placed on a redundant manipulator 190. Referring to FIG. 17, in some embodiments, the redundant manipulator 190 may conveniently be mounted on a monitoring station 192 that may include a display screen 194. Referring to FIG. 18, the redundant manipulator can be controlled to scan through the zygomatic arch 196. Referring to FIG. 19, a redundant manipulator can be controlled to perform a transorbital scan through the eye socket or orbit 198. Referring to FIG. 20, the redundant manipulator can be controlled to scan through the occipital bone 200. Referring to FIG. 21, the redundant manipulator can be controlled to scan through the submandibular 202.

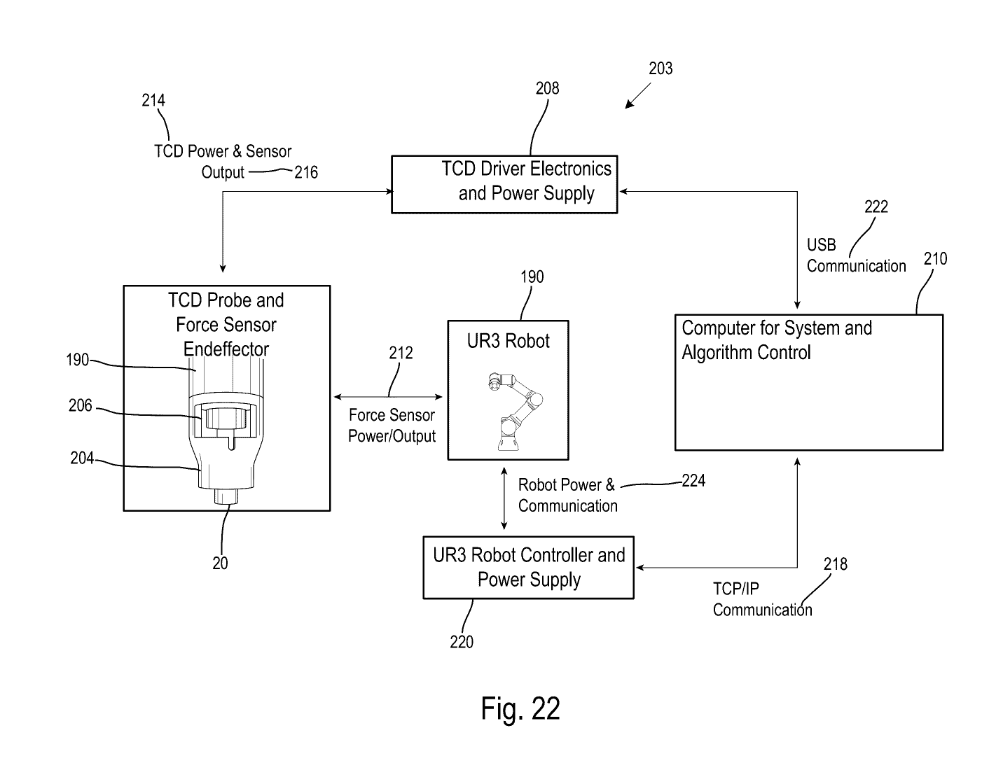

As shown in FIG. 22, in some embodiments, an automated TCD system 203 comprises a redundant manipulator 190 that provides robotic positioning, probe holder 204 with force sensor 206, probe driver board 208, and control computer 210. Some embodiments of the redundant manipulator 190 provide sufficient kinematic precision, have been certified for use around human beings in its workspace, and have settable safety levels to limit velocity and impact forces. These characteristics address safety concerns to allow use with human beings. A probe 20, that may be a Spencer TCD probe, for insonating vessels in a patient or subject, is mounted onto a probe holder 204, called an “endeffector.” The probe holder 204 mounts to the end of the redundant manipulator 190 and has an axial force sensor 206 to monitor directly force applied by the redundant manipulator 190 to the surface being scanned. The force sensor 206 sends force sensor information 212 to the redundant manipulator 190. This force sensor 206 serves both to ensure that enough contact force is made between the probe 20 and the surface being scanned, but is also a second measure of safety to prevent overloading of contact forces. In some embodiments, the probe driver board 208 connects to the probe 20 and provides electronics to send power 214 to the probe to emit ultrasound energy and process the returned sensor output signal 216.

In some embodiments, the control computer 210 connects to the redundant manipulator 190 controller 220 via TCP/IP communication 218 and to the probe driver board 208 by USB 222. The redundant manipulator 190 provides information 224 about such things as its current position, velocity, estimated forces applied to the endeffector, custom sensor readings, and other status information to controller 220, which information 224 is then communicated via TCP/IP 218 to the control computer 210. The probe driver board 208 USB 222 interface provides a method of setting up probe 20 parameters and operation, such as depth, and returns processed data, such as the velocity envelope. The control computer 210 takes all of this information to execute a probe search algorithm and to issue new redundant manipulator 190 commands to move the redundant manipulator. Embodiments may also employ the use of a machine-learning algorithm to emulate the expertise of a trained technician in locating an insonated vessel. In some embodiments, a control computer 210 autonomously controls the probe 20 scanning process, but in other embodiments, a human being may teleoperate the probe 20 scanning process using technology known to those of skill in the art, such as that used in the NeuroArm and DaVinci surgical robots.

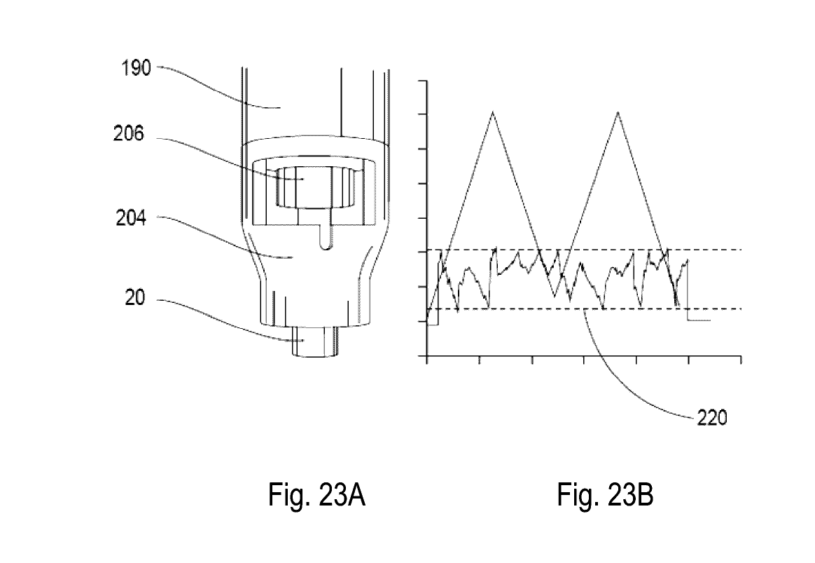

Referring now to FIG. 23A and FIG. 23B, FIG. 23A shows the redundant manipulator 190, force sensor 206, probe holder 204, and probe 20, of FIG. 22. FIG. 23B illustrates the results of tests using this configuration, showing force output controlled to a deadband range 220 of 2 to 10 N.

Integration testing has shown that it is possible to maintain a 125 Hz control loop over the entire system, which is fast enough to read all data from the probe driver board 208, read all status data from the redundant manipulator 190, update a signal processing and planning algorithm, and issue a new motion command to the redundant manipulator 190 at its servo rate.

In some embodiments, a modular snake robot or other robot kinematic configuration with more than six actuated degrees of freedom is used instead of the UR3 as the redundant manipulator.

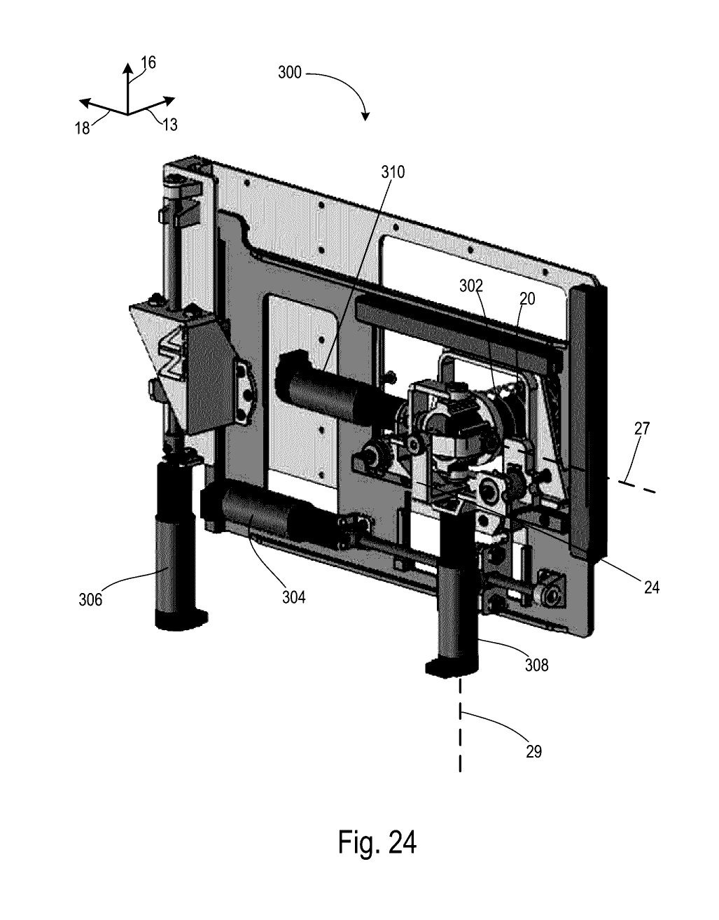

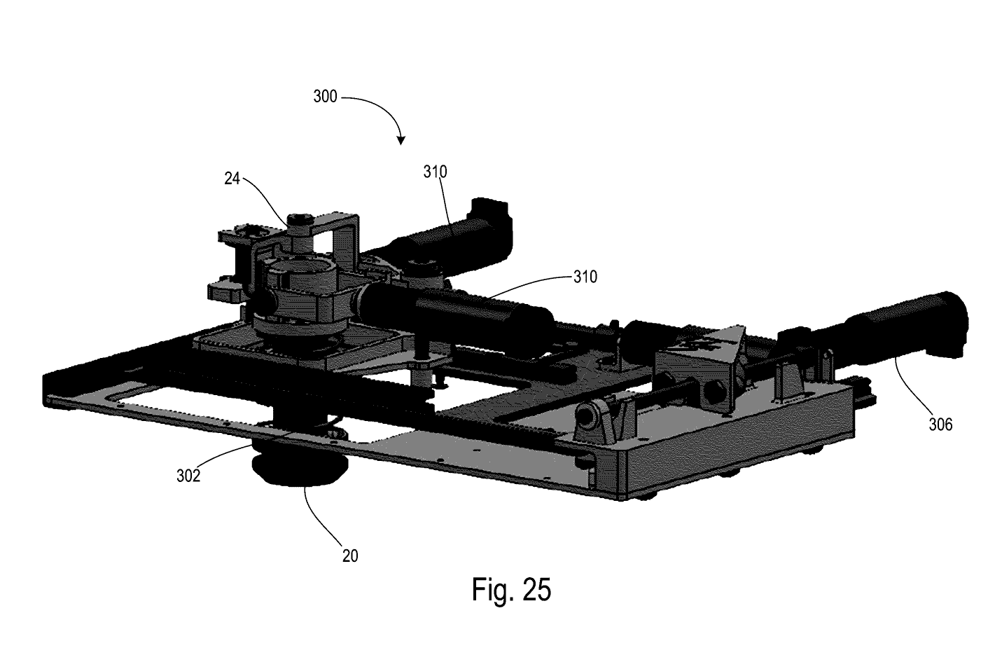

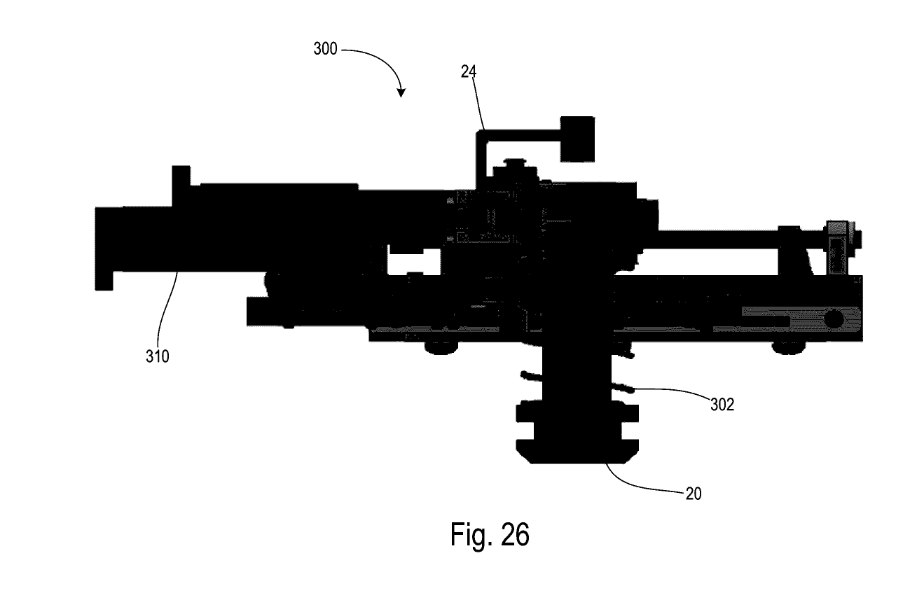

Referring now to FIGS. 24-26, an under actuated system 300 is shown. Such a system has four actuated degrees of freedom in the x,y,pan,tilt axes and a spring providing force along the z axis. In the under actuated system 300 shown, the system has fewer than five actuated degrees of freedom, but is still capable of performing the TCD positioning task. The under actuated system 300 shown is a 4 actuated degree of freedom mechanism that can position and orient the probe 20 in X={x, y, pan, tilt}. In the under actuated system 300, a spring 302 exerts force on the probe 20 along a z-axis 13. In five actuated degree of freedom systems the force exerted by the spring in under actuated system 300 would be actuated by a motor driven mechanism. The gimbal structure 24 allows the probe 20 to be oriented. The force in the z-axis 13 is in effect, monitored by the characterization of the spring constant associated with the spring 302. The actuation in the x-axis 18 is controlled by actuator 304 shown as an electric motor and lead screw. The actuation in the y-axis 16 is controlled by actuator 306 shown as an electric motor and lead screw. The actuation in the pan axis 29 is controlled by motor 308. The actuation in the tilt axis 27 is controlled by motor 310.

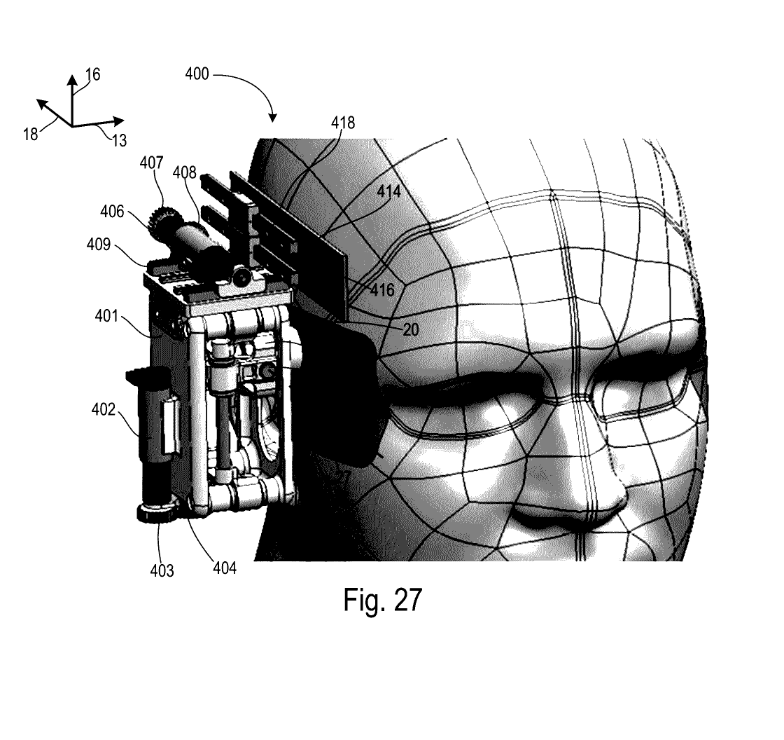

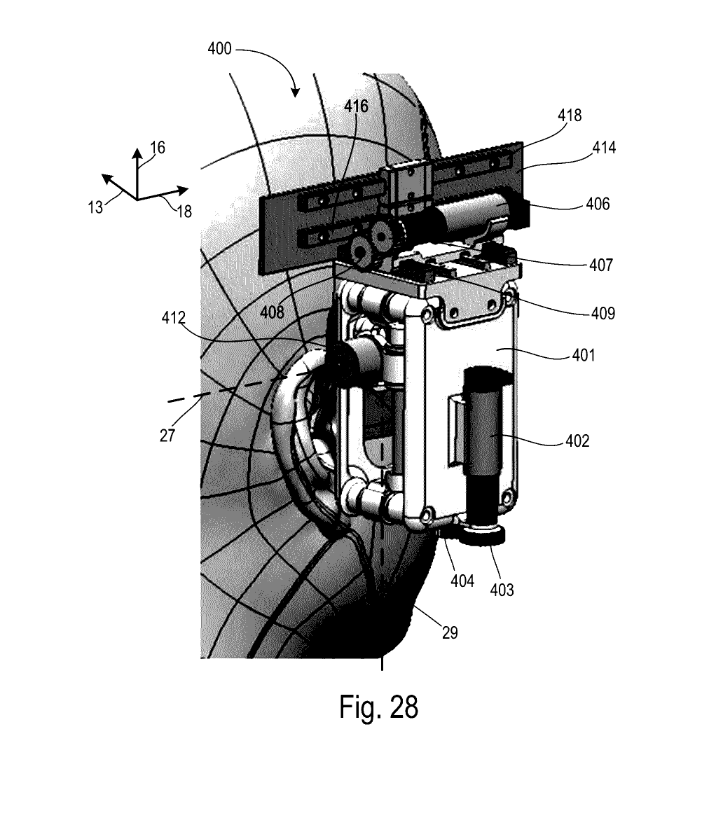

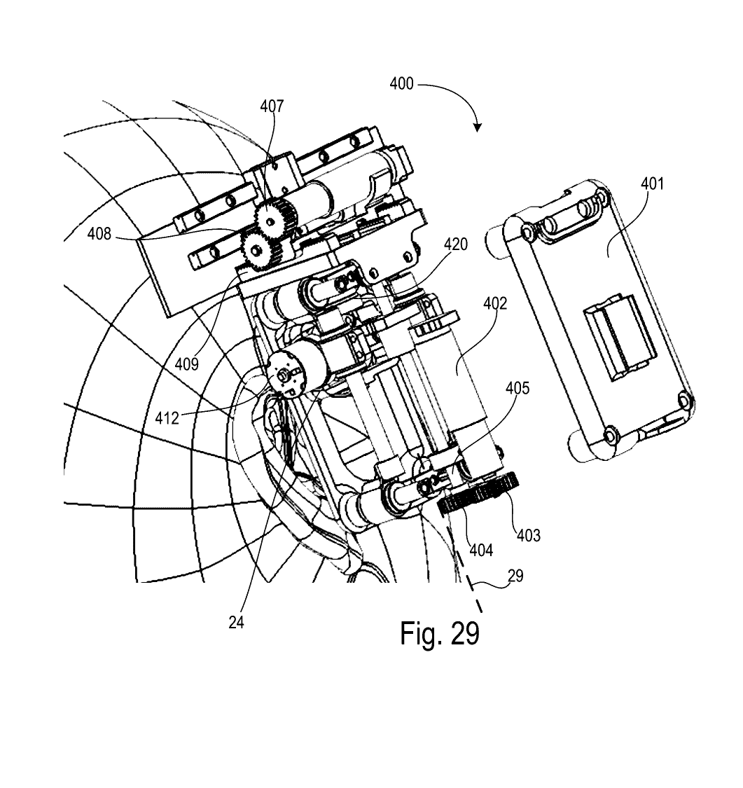

Referring now to FIG. 27, FIG. 28, and FIG. 29 a support structure 400 is shown for the probe 20 and the gimbal structure 24 is shown according to another exemplary embodiment as a prismatic (e.g., Cartesian, rectilinear, etc.) robot. The support structure 400 may, for example, be mounted to a halo or headset 33 worn on the patient's or subject's head or other structure. The support structure 400 includes a cover 401 that covers some of the mechanisms of the support structure 400. In FIG. 29, an exploded view of support structure 400, shows the cover 401 taken off the support structure 400.

A first motor 402 is configured to use a first spur gear 404 to actuate a lead screw 405 mechanism. The first spur gear 403 is coupled to a second spur gear 404 which converts rotational motion of the first motor 402 into linear motion along lead screw 405 to translate the probe 20 along a y-axis 16 (e.g., up and down, bottom of ear to top of ear, etc). A second motor 406 is configured to use a third spur gear 407 coupled to a fourth spur gear 408, which in turn is coupled to a rack and pinion mechanism 409 to translate the probe 20 along a z-axis 13 towards and away from the head of a subject. As shown, in FIG. 28, a third motor 412 and bearing 410 allow for rotation of the gimbal 24 about a tilt axis 27, which in this embodiment, is parallel to x-axis 18. A plate 414 houses two linear rails 416, 418 and allows for mounting of a fourth motor (not shown for clarity) to translate along the x-axis 18 (e.g., forward and backward, ear to eye, etc.). As shown in FIG. 29, a fifth motor 420 allows for controlling rotation of the gimbal 24 about pan axis 29, which, in this embodiment is parallel to y-axis 16, thus completing the necessary degrees of freedom to define five degree of freedom actuated robotic system.

While only a few configurations of a support structure for the probe 20 have been described above and shown in the figures, a person of ordinary skill in the art will understand that many other configurations are possible and that a similar methodology can be used to determine the input forces for the actuators of the support system or from a force-torque sensor to achieve a desired variable stiffness in any direction.

The above used terms, including “attached,” “connected,” “secured,” and the like are used interchangeably. In addition, while certain embodiments have been described to include a first element as being “coupled” (or “attached,” “connected,” “fastened,” etc.) to a second element, the first element may be directly coupled to the second element or may be indirectly coupled to the second element via a third element.

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.” Unless specifically stated otherwise, the term “some” refers to one or more. All structural and functional equivalents to the elements of the various aspects described throughout the previous description that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed as a means plus function unless the element is expressly recited using the phrase “means for.”

It is understood that the specific order or hierarchy of steps in the processes disclosed is an example of illustrative approaches. Based upon design preferences, it is understood that the specific order or hierarchy of steps in the processes may be rearranged while remaining within the scope of the previous description. The accompanying method claims present elements of the various steps in a sample order, and are not meant to be limited to the specific order or hierarchy presented.

The previous description of the disclosed implementations is provided to enable any person skilled in the art to make or use the disclosed subject matter. Various modifications to these implementations will be readily apparent to those skilled in the art, and the generic principles defined herein may be applied to other implementations without departing from the spirit or scope of the previous description. Thus, the previous description is not intended to be limited to the implementations shown herein but is to be accorded the widest scope consistent with the principles and novel features disclosed herein.

What is claimed is:

1. A robotic system for use in scanning a subject, the robotic system comprising:
a probe for emitting energy into the subject; and
a robotic support structure coupled to the probe, the robotic support structure including actuators for moving the probe parallel to a surface of the subject.

2. The robotic system of claim 1, further comprising:
a robotic support structure with five actuated degrees of freedom.

3. The robotic system of claim 1, further comprising:
a robotic support structure with six actuated degrees of freedom.

4. The robotic system of claim 1, further comprising:
a robotic support structure with more than six actuated degrees of freedom.

5. The robotic system of claim 1, further comprising:
a robotic support structure with four actuated degrees of freedom.

6. The robotic system of claim 1, further comprising:
a control computer configured to control movement of the robotic support structure.

7. The robotic system of claim 1, further comprising:
a teleoperated controller configured to control movement of the robotic support structure.

8. The robotic system of claim 1, further comprising:
a hybrid position-force controller configured to control movement of the robotic support structure.

9. The robotic system of claim 1, further comprising a force/torque sensor in contact with the probe.

10. A device configured to interact with a target surface, the device comprising:
a probe configured to interact with the target surface; and
a support structure coupled to the probe for moving the probe relative to the target surface, the support structure comprising:
a hybrid position-force controller that controls movement of the support structure.

11. The device of claim 10, wherein the hybrid position-force controller further comprises:
a spring configured to press the probe against the target surface to maintain contact force passively.

12. The device of claim 11, wherein the hybrid position-force controller further comprises:
a first motor configured to move the probe along a first axis.

13. The device of claim 12, wherein the hybrid position-force controller further comprises:
a second motor configured to move the probe along a second axis.

14. The device of claim 13, wherein the hybrid position-force controller further comprises:
a third motor configured to rotate the probe about a third axis.

15. The device of claim 14, wherein the hybrid position-force controller further comprises:
a fourth motor configured to rotate the probe about a fourth axis.

16. An automated TCD system, comprising:
a TCD probe configured to insonate a vessel of a patient;
a robot mounted to the probe; and
a computer connected to the robot, which computer controls the movement of the robot.

17. The automated TCD system of claim 16, further comprising:
an endeffector with an axial force sensor mounted to the robot in communication with the probe.

18. The automated TCD system of claim 16, wherein the robot is configured to move with at least six actuated degrees of freedom.

19. The automated TCD system of claim 16, wherein the robot is configured to move with exactly five actuated degrees of freedom.

20. The automated TCD system of claim 16, wherein the robot is configured to move with exactly four actuated degrees of freedom.
